(19) United States
(12) Patent Application Publication
(10) Pub. No.: US 2022/0275439 A1
(43) Pub. Date: Sep. 1, 2022

(54) METHODS AND KITS FOR INFERTILITY DIAGNOSTICS

(71) Applicant: Washington State University, Pullman, WA (US)

(72) Inventor: Michael K. Skinner, Pullman, WA (US)

(21) Appl. No.: 17/635,501

(22) PCT Filed: Aug. 14, 2020
(86) PCT No.: PCT/US2020/046455

§ 371 Date: Feb. 15, 2022

Related U.S. Application Data
(60) Provisional application No. 62/887,000, filed on Aug. 15, 2019.

(51) Int. Cl. C12Q 1/6869; C12Q 1/6883

(52) U.S. Cl. CPC C12Q 1/6869; C12Q 1/6883; C12Q 2600/154; C12Q 2600/156

(57) ABSTRACT

Provided herein are methods and kits for providing a likelihood of fertility in a subject. Further, provided herein are methods and kits for determining whether a subject responds to a fertility treatment.

### CROSS REFERENCE

This application claims the benefit of U.S. Provisional Patent Application No. 62/887,000, filed Aug. 15, 2019, which is entirely incorporated herein by reference.

### BACKGROUND

Male fertility over the past century has been observed to have dramatically declined, with recent analysis of data for the past 50 years noting a 50% reduction in male sperm counts. The primary causal factors suggested are environmental exposures influencing testis biology and sperm production. In rodent models, a number of defined toxicants and other exposures promote testis effects associated with a reduction in sperm number. The current estimated infertility range is approximately 15-20% of the human male population. A common strategy for medically assisted reproduction when male factor infertility is identified involves in vitro fertilization and intracytoplasmic sperm injection (ICSI), which are invasive and expensive procedures. In addition to low sperm counts associated with infertility, there is also an increase in idiopathic infertility, which can have normal sperm cohorts and motility. While seminal parameters are commonly used to screen for male factor infertility, the sperm number, motility and shape cannot fully explain the infertility. The development of a clinical diagnostic analysis based on molecular alterations in the sperm would help address this clinical problem.

### SUMMARY OF THE EMBODIMENTS

In an aspect, the present disclosure provides methods for providing a likelihood of fertility in a subject, comprising assaying a nucleic acid sequence from at least a portion of a sperm sample from a subject; detecting a methylation alteration of a portion of said nucleic acid sequence comprised in a differential DNA methylation region (DMR) listed in Table 2, thereby generating an epigenetic profile; and analyzing said epigenetic profile using a computer processor to compare said epigenetic profile with a reference epigenetic profile of a methylation level of at least a portion of a corresponding nucleic acid sequence comprised in said DMR listed in Table 2, wherein when said DMR is DMRMT:1, at least a portion of a nucleic acid sequence comprised in a second DMR, optionally listed in Table 2, is detected and analyzed.

In some embodiments, the methods further comprise determining a likelihood of fertility in said subject at least based in part on said analyzing. In some embodiments, the subject is infertile or has a reduced fertility relative to a normal subject.

In some embodiments, the methods further comprise administering a treatment to said subject. In some embodiments, the treatment comprises performing in vitro fertilization (IVF).

In some embodiments, the treatment comprises performing intracytoplasmic sperm injection (ICSI). In some embodiments, the treatment comprises administering a therapeutic effective amount of follicle stimulating hormone (FSH), or an analog thereof to said subject. In some embodiments, the treatment comprises administering a therapeutic effective amount of human menopausal gonadotropin (hMG), or an analog thereof to said subject.

In some embodiments, the reference epigenetic profile comprises a methylation level of a nucleotide sequence of a fertile subject.

In some embodiments, the detecting comprises measuring an epigenetic alteration of: six or more, ten or more, fifteen or more, twenty or more, thirty or more, forty or more, fifty or more, sixty or more, seventy or more, eighty or more, ninety or more, or one hundred or more DMRs listed in Table 2. In some embodiments, the detecting comprises measuring a methylation alteration of 1-217 DMRs listed in Table 2. In some embodiments, the detecting comprises measuring a methylation alteration of 1-50 DMRs listed in Table 2. In some embodiments, the detecting comprises measuring a methylation alteration of 100-217 DMRs listed in Table 2. In some embodiments, the detecting comprises measuring a methylation alteration of 50-150 DMRs listed in Table 2.

In another aspect, the present disclosure provides methods, comprising: assaying a nucleic acid sequence from at least a portion of a sperm sample from a subject; detecting a methylation alteration of at least a portion of said nucleic acid sequence comprised in a differential DNA methylation region (DMR) listed in Table 3, thereby generating an epigenetic profile; and analyzing said epigenetic profile using a computer processor by comparing to a reference epigenetic profile of methylation level of at least a portion of corresponding nucleic acid sequence comprised in said DMR listed in Table 3.

In some embodiments, when administering a treatment, the methods further comprise determining whether said subject responds to a treatment. In some embodiments, the treatment comprises administering a therapeutic effective amount of follicle stimulating hormone (FSH), or an analog thereof to said subject. In some embodiments, the treatment comprises administering a therapeutic effective amount of human menopausal gonadotropin (hMG), or an analog thereof to said subject.

In some embodiments, wherein when said subject does not respond to said treatment, the methods further comprise performing IVF. In some embodiments, wherein when said subject does not respond to said treatment, the methods further comprise performing ICSI.

In some embodiments, the reference epigenetic profile comprises a methylation level of a nucleotide sequence of a subject that responds to said treatment. In some embodiments, the subject has increased sperm number or sperm motility after receiving said treatment.

In some embodiments, the detecting comprises measuring an epigenetic alteration of: six or more, ten or more, fifteen or more, twenty or more, thirty or more, forty or more, fifty or more DMRs listed in Table 3.

The method of any of claims 15-22, wherein said detecting comprises measuring an epigenetic alteration of 1-56 DMRs listed in Table 3. In some embodiments, the detecting comprises measuring an epigenetic alteration of 1-20 DMRs listed in Table 3. In some embodiments, the detecting comprises measuring an epigenetic alteration of 30-56 DMRs listed in Table 3. In some embodiments, the detecting comprises measuring an epigenetic alteration of 1-35 DMRs listed in Table 3.

In some embodiments, the assaying comprises performing a sequencing analysis, a pyrosequencing analysis, a microarray analysis, or any combination thereof. In some embodiments, the sequencing analysis comprises a methylated DNA immunoprecipitation (MeDIP) sequencing. In some embodiments, the MeDIP comprises using an antibody that binds to a methylated base (mB). In some embodiments, the hmB is 5-methylated base (5-mB). In some embodiments, the 5-hmB is a 5-methylated cytosine (5-mC).

In some embodiments, the epigenetic profile comprises an increased methylation level. In some embodiments, the epigenetic profile comprises a decreased methylation level. In some embodiments, the nucleotide sequence comprises a cytosine phosphate guanine (CpG) region. In some embodiments, the DMRs listed either in Table 2 or Table 3 comprise a CpG density that is less than 10 CpG regions per 100 bp nucleotides.

In some embodiments, the DMRs listed either in Table 2 or Table 3 are produced from about 95% of a genome. In some embodiments, the DMR listed in Table 2 has a range of about 1000 bp to about 50,000 bp nucleotide sequence. In some embodiments, the DMR listed in Table 2 has a range of about 1000 bp to about 4000 bp nucleotide sequence. In some embodiments, the DMR listed in Table 3 has a range of about 1000 bp to about 5000 bp nucleotide sequence. In some embodiments, the DMR listed in Table 3 has a range of about 1000 bp to about 2000 bp nucleotide sequence.

In some embodiments, Table 2 does not overlap with Table 3.

In some embodiments, the methods further comprise obtaining said sperm sample from said subject. In some embodiments, the methods further comprise contacting said nucleic acid sequence with a 5-mC specific antibody. In some embodiments, the methods further comprise contacting said nucleic acid sequence with a bisulfite.

In some embodiments, the subject is a human subject.

In some embodiments, the methods further comprise transmitting a result via a communication medium. In some embodiments, the result comprises an epigenetic profile, a reference epigenetic profile, or both.

In another aspect, the present disclosure provides a kit, comprising: bisulfite; a plurality of primers configured to detect a differential DNA methylation region (DMR) listed in Table 2 or Table 3; and a microarray chip or a DNA sequencing kit.

In another aspect, the present disclosure provides a computer-readable medium comprising machine-executable code that, upon execution by a computer processor, implements a method for determining a likelihood of fertility in a subject, comprising: assaying a nucleic acid sequence from at least a portion of a sperm sample from a subject; detecting a methylation alteration of a portion of said nucleic acid sequence comprised in a differential DNA methylation region (DMR) listed in Table 2, thereby generating an epigenetic profile; and analyzing said epigenetic profile using a computer processor to compare said epigenetic profile with a reference epigenetic profile of a methylation level of at least a portion of corresponding nucleic acid sequence comprised in said DMR listed in Table 2, wherein when said DMR is DMRMT:1, at least a portion of a nucleic acid sequence comprised in a second DMR, optionally listed in Table 2, is optionally detected, and is analyzed.

In another aspect, the present disclosure provides a computer-readable medium comprising machine-executable code that, upon execution by a computer processor, implements a method for determining whether a subject responds to a treatment, comprising: assaying a nucleic acid sequence from at least a portion of a sperm sample from a subject; detecting a methylation alteration of at least a portion of said nucleic acid sequence comprised in a differential DNA methylation region (DMR) listed in Table 3, thereby generating an epigenetic profile; and analyzing said epigenetic profile using a computer processor by comparing to a reference epigenetic profile of methylation status of at least a portion of corresponding nucleic acid sequence comprised in said DMR listed in Table 3.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by references to the same extent as if each individual publication, patent, or patent application was specifically and individually indicated to be incorporated by reference. To the extent publications or patents or patent applications incorporated by reference contradict the disclosure contained in the specification, the specification is intended to supersede and/or take precedence over any such contradictory material.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A-F shows infertility patients' semen and sperm parameters upon recruitment (Pre-Conc 0) prior to FSH therapeutic treatment (Pre-Conc 1) and after 3 months of treatment (Post-Conc 2) for individual patients listed. Sample analyses for all patients are presented in (A) Semen concentration, (B) Percent motility sperm, and (C) Total motility count (TMC) (semen volume×concentration×motility). Infertility patients responding with >2-fold change following treatment are presented, (D) Semen concentration, (E) Percent motility sperm, and (F) TMC. The y-axis is magnitude of change between collections.

FIG. 2A-D shows the DMR identifications. (A) Fertility vs Infertility Sperm DMR Analysis. The number of DMRs found using different p-value cutoff thresholds. The all window column shows all DMRs. The multiple window column shows the number of DMRs containing at least two adjacent significant windows and the number of DMRs with each specific number of significant windows at a p-value threshold of 1e-05. (B) Infertility patient responder vs non-responder sperm DMRs. The number of DMRs found using different p-value cutoff thresholds. The all window column shows all DMRs. The multiple window column shows the number of DMRs containing at least two significant windows. The number of DMRs with each specific number of significant windows at a p-value threshold of 1e-05. (C) Venn diagram DMR signature for fertile vs infertile p<1e-05 and DMR signature responder vs. non-responder at p<1e-05 and p<0.001. (D) DMR associated gene categories.

FIG. 3A-F shows the DMR genomic characteristics. (A) Chromosomal Locations of Fertility vs Infertility DMR Analysis. The DMR locations on the individual chromosomes. All DMRs at a p-value threshold of p<1e-05 are shown with the arrowhead and clusters of DMRs with the boxes. (B) Responder DMR Signature Chromosomal Locations. The DMR locations (arrowhead) and clusters of DMRs (box) on the individual chromosomes. All DMRs at a p-value threshold of p<1e-05 are shown. (C) DMR CpG density in the Fertility vs Infertility DMRs. The number of DMRs at different CpG densities. All DMRs at a p-value threshold of p<1e-05 are shown. (D) The Responder signature DMR CpG density (number per 100 bp). The number of DMRs at different CpG densities are presented. All DMRs at a p-value threshold of 1e-05 are shown. (E) Fertility vs Infertility DMR lengths in kilobases. All DMRs at a p-value threshold of 1e-05 are shown. (F) The Responder signature DMRs size in kilobases. All DMRs at a p-value threshold of 1e-05 are shown.

FIG. 4A-D shows the Principal component analysis. (A) Fertility vs Infertility DMR Principal Component Analysis for Individuals. The samples are plotted by the first three principal components. The underlying data is the RPKM read depth for the DMRs. (B) Fertility vs Infertility DMR Principal Component Analysis for Individuals. The samples are plotted by the first three principal components. The underlying data is the RPKM read depth for the DMRs. Selection failure correlations for fertile and infertile patients not used to generate the epigenetic signature. PCA Infertile vs Fertile p<1e-5. (C) Responder and non-responder PCA analysis for DMRs at p<1e-05. The first three principal components used are indicated. The underlying data is the RPKM read depth for all DMRs. (D) The number of DMR for fertility versus infertility comparison for all permutation analyses. The vertical line shows the number of DMR found in the original analysis. All DMRs are defined using an edgeR p-value threshold of p<1e-05.

FIG. 5 shows a computer system that is programmed or otherwise configured to implement methods of the present disclosure, such as assaying nucleic acid sequence(s), detecting methylation alteration, and analyzing epigenetic profile(s) in samples, in accordance with some embodiments.

### DETAILED DESCRIPTION

The primary source of increased male factor infertility and decline in seminal parameters appear to be environmental exposures. This includes a variety of toxicants, endocrine disruptors, abnormal nutrition, smoking and alcohol, and stress. Animal models have demonstrated the direct actions of a number of environmental toxicants to reduce sperm numbers and promote testis disease and male infertility. Various human male exposures also have been shown to associate with poor sperm parameters and male infertility. The primary molecular actions considered involve environmental epigenetics.

Epigenetics is defined as “molecular factors or processes around DNA that regulate germline activity independent of DNA sequence and are mitotically stable”. One of the principal epigenetic processes involved in sperm abnormalities is DNA methylation. Cytosine methylation at CpG sites can alter gene expression, and within sperm these sites are associated with reduced fertility and promotion of disease in offspring. Altered sperm methylation has been shown to be a biomarker for environmental exposures that associate with various pathologies later in life. Although altered histone retention following protamine replacement in sperm and non-coding RNAs have also been shown to associate with male infertility, the primary epigenetic biomarker investigated in the current study involves DNA methylation.

Animal models initially demonstrated a correlation with sperm DNA methylation and male infertility. Human studies have also demonstrated a decreased fecundity associated with sperm DNA methylation alterations. A sperm DNA methylation biomarker assay has been developed and validated, which uses a microarray approach to assess CpG islands within the genome. Although this analysis only investigates approximately 1% of the genome, it has been shown to be useful in analysis of sperm DNA methylation in a clinical setting. Subsequently, studies with in vitro fertilization (IVF) applications have used measurement of DNA methylation with this biomarker analysis to assess male infertility prior to assisted reproduction. Since this previous analysis only examined a limited amount of the genome (i.e. <1%), the current study was designed to investigate a more genome-wide approach using low density CpG regions (i.e. 95% genome) to examine alterations in sperm DNA methylation.

A promising approach for the clinical therapy of male infertility is the use of endocrine therapeutics, similar to what is used in the female. For example, observations suggest a beneficial effect of FSH treatment on spontaneous pregnancy and live birth rate in men with idiopathic male factor infertility. Therapy with exogenous follicle stimulating hormone (FSH) is achieved by administration of urinary or recombinant FSH preparations or human menopausal gonadotropin (hMG) preparations, with the latter providing both FSH activity and luteinizing hormone (LH) activity. In women, FSH therapy is successfully used to stimulate oogenesis, and a similar approach would be expected to induce spermatogenesis. Due to the variable response within the infertile population, a diagnostic test to assess a responder versus non-responder individuals would be expected to significantly enhance the utility of FSH therapeutics.

All clinical therapeutic studies have identified responder and non-responder subpopulations. Those that are efficacious for the majority of the population are generally not as concerned with the non-responder population. When the majority of the disease population does not respond, such as immune therapy for arthritis, the advancement of a molecular diagnostic for the responder versus the non-responder population would be very useful in the management of the disease. Although a number of disease biomarkers or diagnostics have been identified for disease, few have been observed for specific responder versus non-responder subpopulations.

### Definitions

The following are definitions of terms that may be used in the present specification. The initial definition provided for a group or term herein applies to that group or term throughout the present specification individually or as part of another group, unless otherwise indicated.

Additionally, it will be understood that any list of such candidates or alternatives is merely illustrative, not limiting, unless implicitly or explicitly understood or stated otherwise.

As used in this specification and the appended claims, the singular forms “a”, “an” and “the” include plural referents unless the content clearly dictates otherwise.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

All scientific and technical terms used in this application have meanings commonly used in the art unless otherwise specified. As used in this application, the following words or phrases have the meanings specified.

The term “subject,” as used herein, may be any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent or adult animals. Humans can be more than about: 1, 2, 5, 10, 20, 30, 40, 50, 60, 65, 70, 75, or about 80 years of age. The subject may have or be suspected of having a condition or a disease, such as infertility or idiopathic infertility. The subject may be a patient, such as a patient being treated for a condition or a disease, such as an infertility patient. The subject may be predisposed to a risk of developing a condition or a disease such as infertility. The subject may be in remission from a condition or a disease, such as an infertility patient. The subject may be healthy or normal without any infertility issues.

The term sensitivity, or true positive rate, can refer to a test's ability to identify a condition correctly. For example, in a diagnostic test, the sensitivity of a test is the proportion of patients known to have the disease or condition, who will test positive for it. In some cases, this is calculated by determining the proportion of true positives (i.e. patients who test positive who have the disease) to the total number of individuals in the population with the condition (i.e., the sum of patients who test positive and have the condition and patients who test negative and have the condition).

“Infertility” generally refers to the inability of a sexually active, non-contracepting couple to achieve pregnancy in at least one year. The methods of the present disclosure relate to infertility that is attributable to the male subject. “Infertility” as used herein may also include subfertility which relates to reduced fertility compared to a normal subject that has no fertility issues for any period of time. Causes of infertility or reduced fertility in male subjects may include, for example, abnormal sperm production or function, problems with the delivery of sperm, overexposure to certain environmental factors such as pesticides, radiation, medication, cigarette smoke, etc., and damage related to cancer and its treatment.

“Epimutation,” “epigenetic modification,” as used herein generally refer to modifications of cellular DNA that affect gene expression without altering the DNA sequence. The epigenetic modifications are both mitotically and meiotically stable, i.e. after the DNA in a cell (or cells) of an organism has been epigenetically modified, the pattern of modification persists throughout the lifetime of the cell and is passed to progeny cells via both mitosis and meiosis. Therefore, with the organism's lifetime, the pattern of DNA modification and consequences thereof, remain consistent in all cells derived from the parental cell that was originally modified. Further, if the epigenetically modified cell undergoes meiosis to generate gametes (e.g. sperm), the pattern of epigenetic modification is retained in the gametes and thus inherited by offspring. In other words, the patterns of epigenetic DNA modification are transgenerationally transmissible or inheritable, even though the DNA nucleotide sequence per se has not been altered or mutated. Without being bound by theory, it is believed that enzymes known as methyltransferases shepherd or guide the DNA through the various phases of mitosis or meiosis, reproducing epigenetic modification patterns on new DNA strands as the DNA is replicated. Exemplary epigenetic modifications include, but are not limited, to DNA methylation, histone modifications, chromatin structure modifications, and non-coding RNA modifications, etc.

Further, the term “epigenetic modification” as used herein, may be any covalent modification of a nucleic acid base. In some cases, a covalent modification may comprise (i) adding a methyl group, a hydroxymethyl group, a carbon atom, an oxygen atom, or any combination thereof to one or more bases of a nucleic acid sequence, (ii) changing an oxidation state of a molecule associated with a nucleic acid sequence, such as an oxygen atom, or (iii) a combination thereof. A covalent modification may occur at any base, such as a cytosine, a thymine, a uracil, an adenine, a guanine, or any combination thereof. In some cases, an epigenetic modification may comprise an oxidation or a reduction. A nucleic acid sequence may comprise one or more epigenetically modified bases. An epigenetically modified base may comprise any base, such as a cytosine, a uracil, a thymine, adenine, or a guanine. An epigenetically modified base may comprise a methylated base, a hydroxymethylated base, a formylated base, or a carboxylic acid containing base or a salt thereof. An epigenetically modified base may comprise a 5-methylated base, such as a 5-methylated cytosine (5-mC). An epigenetically modified base may comprise a 5-hydroxymethylated base, such as a 5-hydroxymethylated cytosine (5-hmC). An epigenetically modified base may comprise a 5-formylated base, such as a 5-formylated cytosine (5-fC). An epigenetically modified base may comprise a 5-carboxylated base or a salt thereof, such as a 5-carboxylated cytosine (5-caC). In some cases, an epigenetically modified base may comprise a methyltransferase-directed transfer of an activated group (mTAG).

Epigenetic modifications may be caused by exposure to any of a variety of factors, examples of which include but are not limited to: chemical compounds e.g. endocrine disruptors such as vinclozolin; chemicals such as those used in the manufacture of plastics e.g. bispheol A (BPA); bis(2-ethylhexyl)phthalate (DEHP); dibutyl phthalate (DBP); insect repellants such as N, N-diethyl-meta-toluamide (DEET); pyrethroids such as permethrin; various polychlorinated dibenzodioxins, known as PCDDs or dioxins e.g. 2,3,7,8-tetrachlorodibenzo-p-dioxin (TCDD); extreme conditions such as abnormal nutrition, starvation, chemotherapeutic agents which include alkylating agents such as ifosfamide and cyclophosphamide, anthracyclines such as daunorubicin and doxorubicine, taxanes such as paclitaxel and docetaxel, epothilones, histone deacetylase inhibitors, topoisomerase inhibitors, kinase inhibitors such as gefitinib, platinum-based agents such as cisplatin, retinoids, and vinca alkaloids, etc.

Methylation level, as used herein, generally refers to a percentage of nucleotides of a nucleotide sequence that are methylated. DNA methylation is an epigenetic mechanism that occurs when a methyl group is added onto the C5 position of cytosine, thereby modifying gene function and affecting gene expression. Most DNA methylation occurs at cytosine residues that precede guanine residues, called CpG dinucleotides, which tend to cluster in DNA domains known as CpG islands. Methylation level may be measured on a genome wide basis of any CpG containing regions. Methylation alteration, as used herein, generally refers to an increase or decrease of a percentage of nucleotides of a nucleotide sequence that are methylated.

The term “nucleic acid sequence” as used herein may comprise DNA or RNA. In some cases, a nucleic acid sequence may comprise a plurality of nucleotides. In some cases, a nucleic acid sequence may comprise an artificial nucleic acid analogue. In some cases, a nucleic acid sequence comprising DNA, may comprise cell-free DNA, cDNA, fetal DNA, or maternal DNA. In some cases, a nucleic acid sequence may comprise miRNA, shRNA, or siRNA.

The term “fragment,” as used herein, may be a portion of a sequence, a subset that may be shorter than a full length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, 50 amino acids in length. A fragment may be less than about: 20, 30, 40, 50 oligonucleotides in length.

The term “biological sample” generally refers to any fluid or cellular sample or mixture thereof obtained from a living organism. The biological sample may be a reproductive sample, such as an egg or a sperm. Exemplary biological samples may include tissue biopsy, serum, plasma, and buccal cells.

The term “sequencing” as used herein, may comprise bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore DNA sequencing, shot gun sequencing, RNA sequencing, Enigma sequencing, or any combination thereof.

A “plurality” as used herein generally refers to two or more DMRs, for example, two, three, four, five, six, and every integer up to and including all of the DMRs listed in table 2 or 3. A plurality may also refer to two or more DMRs listed in table 2 or 3 and every integer up to and including all DMRs listed in table 2 or 3.

The term “responder” as used herein, generally relates to patients for which the predicted response to the treatment/biological drug is positive, i.e., increasing in sperm numbers (sperm concentration), sperm motility, or both. Sperm numbers or sperm concentration may be measured by any suitable known methods. Further, sperm motility may be measured any suitable known methods. Similarly, the term “non-responder” as used herein, generally relates to patients for which the predicted response to the treatment/biological drug is negative.

The term “predicted response” or similar, as used herein refers to the determination of the likelihood that the patient will respond either favorably or unfavorably to a given therapy/biological drug. Especially, the term “prediction”, as used herein, relates to an individual assessment of any parameter that can be useful in determining the evolution of a patient. As it will be understood by those skilled in the art, the prediction of the clinical response to the treatment with a biological drug, although preferred to be, need not be correct for 100% of the subjects to be diagnosed or evaluated. The term, however, requires that a statistically significant portion of subjects can be identified as having an increased probability of having a positive response. Whether a subject is statistically significant can be determined without further effort by the person skilled in the art using various well known statistic evaluation tools, e.g., determination of confidence intervals, p-value determination, Student's t-test, Mann-Whitney test, etc. Preferred confidence intervals are at least 50%, at least 60%, at least 70%, at least 80%, at least 90% at least 95%. The p-values are, preferably, 0.2, 0.1 or 0.05.

Patients achieving complete or partial response are considered “responders”, and all other patients are considered “non-responders”.

Provided herein are differential DNA methylation regions (DMRs) which are useful for the identification of male subjects are infertile (Table 2). Also provided are DMRs which are useful for the identification of infertile male subjects who are responders to FSH therapy (Table 3). The tables provide the DMR name, chromosome location, start and stop base pair location, length in base pair (bp), number of significant windows (100 bp), p-value, number of CpG sites, CpG sites per 100 bp, and DMR associated gene symbol (annotation). Each start site corresponds to the GRCh38 reference genome (as originally released in December 2013) that is well known in the art. It is also known in the art that any “patches” to the GRCh38 genome that were subsequently released do not change the chromosomal coordinates of the reference genome. In the context of the present disclosure, the specific sequence where a DMR is located is not critical as methylation does not affect the underlying sequence. Disclosed herein are specific locations within the genome that contain differential methylation (irrespective of the underlying genome sequence) that is indicative of infertility or responsiveness to FSH therapy. Thus, the DMR may be located in within a sequence that is about 50% identical to any of the sequences listed in Table 2 or Table 3, e.g. at least about 60%, 65%, 70%, 75%, 80%, 85%, 90%, 95%, 96%, 97%, 98%, or 99% identical.

In some embodiments, the level of methylation at a DMR is increased or decreased by at least about 10% as compared to a control or a reference sample collected from a normal subject without infertility. In some embodiments, the methylation level is determined by a cytosine. In some embodiments, the DMRs are associated with certain genes in an individual. In some embodiments, the DMRs are associated with certain CpG loci. The CpG loci may be located in the promoter region of a gene, in an intron or exon of a gene or located near the gene in a patient's genomic DNA. In an alternate embodiment, the CpG may not be associated with any known gene or may be located in an intergenic region of a chromosome. In some embodiments, the CpG loci may be associated with one or more than one gene.

In some instances, the DMRs described herein are found in CpG desert regions of the genome, e.g. a CpG density of about 10% or less or a mean around two CpG per 100 base pairs. Due to the evolutionary conservations of CpG clusters in a CpG desert, these are likely epigenetic regulatory sites. Additional genomic features of characteristic of ECRs are described in U.S. Patent Publication 2013/0226468 incorporated herein by reference. Those of skill in the art will recognize that the “%” of a sequence of interest (e.g. CpG) means that the sequence occurs the indicated number of times per 100 base pairs analyzed, e.g. 15% or less CpG means that the dinucleotide sequence C followed by G occurs at most 15 times per 100 base pairs within a DNA segment that is analyzed. Analyses are usually carried out by iterative analysis of consecutively overlapping sequences within a large DNA molecule of interest, e.g. a chromosome, a section of a chromosome, etc.

The DMRs provided herein allow for determining if a male subject is infertile comprising detecting a methylation alteration of a portion of said nucleic acid sequence comprised in a differential DNA methylation region (DMR) listed in Table 2, thereby generating an epigenetic profile; and analyzing said epigenetic profile using a computer processor to compare said epigenetic profile with a reference epigenetic profile of a methylation level of at least a portion of corresponding nucleic acid sequence comprised in said DMR listed in Table 2, wherein when said DMR is DMRMT:1, a second DMR is detected and analyzed. For example, if a DMR from Table 2 comprises 2000 bp nucleotides, in some embodiments, at least about 50%, about 60%, about 70%, about 80%, about 90%, or about 100% of the 2000 bp nucleotides may be measured to determine the methylation level. The DMR named DMRMT:1 as listed in Table 2 is associated with genes, such as RNR1 as listed in Table 2. In some embodiments, the second DMR is selected from Table 2. In some embodiments, the second DMR is not selected from Table 2.

In some embodiments, the methylation level of each DMR contained in a subject's epigenetic profile is measured by methods disclosed herein, such as methylated DNA immunoprecipitation (MeDIP) sequencing. In some embodiments, the methylation level of a corresponding DMR contained in a reference epigenetic profile from a healthy normal subject is measure by methods disclosed herein or obtained from public information. Then the methylation levels of DMRs are compared between the epigenetic profile and the reference epigenetic profile using any suitable methods including suitable computer programs.

In some embodiments, the epigenetic profile comprises a plurality of DMRs selected from the group listed in table 2. In other embodiments, the epigenetic modification comprises all of the DMRs listed in table 2. In some embodiments, the epigenetic profile comprises at least two DMRs listed in Table 2. In some embodiments, the epigenetic profile comprises six or more, ten or more, fifteen or more, twenty or more, thirty or more, forty or more, fifty or more, sixty or more, seventy or more, eighty or more, ninety or more, or one hundred or more DMRs listed in Table 2.

TABLE 2
Fertility versus infertility DMRs with various genomic features

| | | | | | # Sig | | |
|---|---|---|---|---|---|---|---|
| DMR Name | Chr | Start | Stop | Length | Win | minP | maxLFC |
| DMR1: 629001 | 1 | 629001 | 635000 | 6000 | 4 | 1.10E−08 | −1.3540969 |
| DMR1: 4712001 | 1 | 4712001 | 4713000 | 1000 | 1 | 7.02E−06 | −1.3546468 |
| DMR1: 24099001 | 1 | 24099001 | 24101000 | 2000 | 1 | 5.74E−06 | −1.3031799 |
| DMR1: 121860001 | 1 | 121860001 | 121861000 | 1000 | 1 | 1.84E−06 | −1.8326274 |
| DMR1: 144475001 | 1 | 144475001 | 144476000 | 1000 | 1 | 2.11E−07 | −1.3557747 |
| DMR1: 153464001 | 1 | 153464001 | 153465000 | 1000 | 1 | 5.26E−06 | −1.2143061 |
| DMR1: 156732001 | 1 | 156732001 | 156733000 | 1000 | 1 | 1.55E−06 | −1.3923279 |
| DMR1: 204834001 | 1 | 204834001 | 204835000 | 1000 | 1 | 2.40E−06 | 0.9757061 |
| DMR1: 226959001 | 1 | 226959001 | 226960000 | 1000 | 1 | 7.60E−06 | −0.7873646 |
| DMR2: 3591001 | 2 | 3591001 | 3592000 | 1000 | 1 | 8.48E−06 | −1.7009923 |
| DMR2: 10913001 | 2 | 10913001 | 10915000 | 2000 | 1 | 2.97E−06 | −2.02485 |
| DMR2: 87091001 | 2 | 87091001 | 87092000 | 1000 | 1 | 3.41E−06 | 0.8659935 |
| DMR2: 87348001 | 2 | 87348001 | 87350000 | 2000 | 1 | 1.87E−06 | −1.2930736 |
| DMR2: 87405001 | 2 | 87405001 | 87432000 | 27000 | 6 | 1.36E−06 | −1.7393104 |
| DMR2: 101342001 | 2 | 101342001 | 101344000 | 2000 | 1 | 1.10E−07 | −2.5359785 |
| DMR2: 104368001 | 2 | 104368001 | 104370000 | 2000 | 1 | 7.64E−06 | −1.2924014 |
| DMR2: 131646001 | 2 | 131646001 | 131647000 | 1000 | 1 | 3.57E−06 | −1.2582149 |
| DMR2: 201858001 | 2 | 201858001 | 201859000 | 1000 | 1 | 1.94E−06 | 0.9091697 |
| DMR2: 211358001 | 2 | 211358001 | 211359000 | 1000 | 1 | 5.28E−06 | 0.931714 |
| DMR2: 225582001 | 2 | 225582001 | 225583000 | 1000 | 1 | 2.80E−06 | −2.2744394 |
| DMR3: 112966001 | 3 | 112966001 | 112968000 | 2000 | 1 | 1.45E−07 | 0.7662919 |
| DMR3: 125912001 | 3 | 125912001 | 125914000 | 2000 | 1 | 7.15E−06 | −1.6588343 |
| DMR3: 150778001 | 3 | 150778001 | 150779000 | 1000 | 1 | 8.51E−07 | −1.6179423 |
| DMR3: 176844001 | 3 | 176844001 | 176845000 | 1000 | 1 | 6.37E−09 | 1.003399 |
| DMR3: 184170001 | 3 | 184170001 | 184171000 | 1000 | 1 | 5.67E−06 | −2.1218771 |
| DMR3: 188433001 | 3 | 188433001 | 188434000 | 1000 | 1 | 1.84E−06 | 0.7498256 |
| DMR4: 53001 | 4 | 53001 | 57000 | 4000 | 1 | 4.39E−07 | −2.391171 |
| DMR4: 747001 | 4 | 747001 | 749000 | 2000 | 1 | 8.83E−06 | −1.9049842 |
| DMR4: 3809001 | 4 | 3809001 | 3811000 | 2000 | 1 | 1.96E−06 | −2.285143 |
| DMR4: 49270001 | 4 | 49270001 | 49274000 | 4000 | 1 | 1.80E−08 | −1.4604032 |
| DMR4: 49275001 | 4 | 49275001 | 49278000 | 3000 | 1 | 1.05E−07 | −1.5488942 |
| DMR4: 49279001 | 4 | 49279001 | 49284000 | 5000 | 1 | 9.37E−06 | −1.721407 |
| DMR4: 49291001 | 4 | 49291001 | 49303000 | 12000 | 2 | 7.79E−06 | −1.5240589 |
| DMR4: 49313001 | 4 | 49313001 | 49318000 | 5000 | 1 | 3.32E−06 | −1.2186361 |
| DMR4: 49319001 | 4 | 49319001 | 49325000 | 6000 | 1 | 4.35E−06 | −1.4846204 |
| DMR4: 49510001 | 4 | 49510001 | 49520000 | 10000 | 1 | 3.55E−06 | −1.1945741 |
| DMR4: 68471001 | 4 | 68471001 | 68472000 | 1000 | 1 | 2.45E−06 | 1.361757 |
| DMR4: 69599001 | 4 | 69599001 | 69600000 | 1000 | 1 | 4.86E−06 | 1.1306075 |
| DMR4: 185384001 | 4 | 185384001 | 185385000 | 1000 | 1 | 6.27E−06 | 1.0448613 |
| DMR4: 186889001 | 4 | 186889001 | 186890000 | 1000 | 1 | 2.89E−06 | −1.7304782 |
| DMR4: 190021001 | 4 | 190021001 | 190023000 | 2000 | 1 | 8.41E−06 | −0.9758405 |
| DMR5: 258001 | 5 | 258001 | 262000 | 4000 | 1 | 1.75E−07 | −2.7261411 |
| DMR5: 1570001 | 5 | 1570001 | 1571000 | 1000 | 1 | 7.01E−06 | −1.4101567 |
| DMR5: 12433001 | 5 | 12433001 | 12434000 | 1000 | 1 | 9.37E−06 | 0.8019226 |
| DMR5: 17581001 | 5 | 17581001 | 17588000 | 7000 | 1 | 1.64E−06 | −2.6252202 |
| DMR5: 17589001 | 5 | 17589001 | 17600000 | 11000 | 5 | 2.39E−06 | −2.6544095 |
| DMR5: 36783001 | 5 | 36783001 | 36784000 | 1000 | 1 | 1.90E−06 | −1.5416817 |
| DMR5: 46622001 | 5 | 46622001 | 46623000 | 1000 | 1 | 6.44E−06 | −1.5676853 |
| DMR6: 105831001 | 6 | 105831001 | 105832000 | 1000 | 1 | 2.05E−07 | 1.5144618 |
| DMR6: 132600001 | 6 | 132600001 | 132602000 | 2000 | 1 | 5.81E−06 | 1.5140375 |
| DMR6: 150337001 | 6 | 150337001 | 150338000 | 1000 | 1 | 1.23E−06 | −1.2839276 |
| DMR6: 163194001 | 6 | 163194001 | 163197000 | 3000 | 1 | 4.93E−06 | −2.1415117 |
| DMR6: 167731001 | 6 | 167731001 | 167732000 | 1000 | 1 | 1.71E−07 | −1.9579585 |
| DMR6: 170138001 | 6 | 170138001 | 170139000 | 1000 | 1 | 6.23E−06 | −1.4198044 |
| DMR7: 636001 | 7 | 636001 | 638000 | 2000 | 1 | 4.76E−06 | −2.1159539 |
| DMR7: 2577001 | 7 | 2577001 | 2579000 | 2000 | 2 | 8.65E−07 | −2.5038122 |
| DMR7: 10779001 | 7 | 10779001 | 10780000 | 1000 | 1 | 8.01E−07 | −2.2960801 |
| DMR7: 37256001 | 7 | 37256001 | 37257000 | 1000 | 1 | 9.30E−07 | 1.0459778 |
| DMR7: 58104001 | 7 | 58104001 | 58120000 | 16000 | 1 | 2.60E−07 | −1.5731667 |
| DMR7: 64407001 | 7 | 64407001 | 64408000 | 1000 | 1 | 3.09E−07 | −1.5558383 |
| DMR7: 85091001 | 7 | 85091001 | 85092000 | 1000 | 1 | 5.01E−06 | 0.9594642 |
| DMR7: 128586001 | 7 | 128586001 | 128587000 | 1000 | 1 | 9.46E−06 | −0.8221203 |
| DMR7: 155788001 | 7 | 155788001 | 155790000 | 2000 | 1 | 4.97E−06 | −1.6163775 |
| DMR8: 12541001 | 8 | 12541001 | 12542000 | 1000 | 1 | 2.30E−06 | −1.2106429 |
| DMR8: 23248001 | 8 | 23248001 | 23249000 | 1000 | 1 | 9.44E−06 | −1.3344564 |
| DMR8: 42053001 | 8 | 42053001 | 42054000 | 1000 | 1 | 6.30E−08 | −1.9852231 |
| DMR8: 46103001 | 8 | 46103001 | 46106000 | 3000 | 1 | 1.92E−06 | −1.1795439 |
| DMR8: 46257001 | 8 | 46257001 | 46261000 | 4000 | 1 | 1.70E−07 | −1.7683408 |
| DMR8: 52388001 | 8 | 52388001 | 52389000 | 1000 | 1 | 8.83E−06 | −1.8314409 |
| DMR8: 85642001 | 8 | 85642001 | 85644000 | 2000 | 1 | 5.21E−06 | −2.3394436 |
| DMR8: 85645001 | 8 | 85645001 | 85664000 | 19000 | 4 | 1.93E−07 | −2.464287 |
| DMR8: 85714001 | 8 | 85714001 | 85766000 | 52000 | 12 | 1.17E−08 | −3.0057258 |
| DMR8: 85767001 | 8 | 85767001 | 85781000 | 14000 | 2 | 2.49E−07 | −2.6820478 |
| DMR8: 85782001 | 8 | 85782001 | 85830000 | 48000 | 13 | 3.62E−09 | −2.8895738 |
| DMR8: 112511001 | 8 | 112511001 | 112512000 | 1000 | 1 | 1.79E−06 | −1.1234341 |
| DMR8: 133772001 | 8 | 133772001 | 133774000 | 2000 | 1 | 1.84E−06 | −1.0463593 |
| DMR9: 3536001 | 9 | 3536001 | 3537000 | 1000 | 1 | 4.61E−06 | −1.5886032 |
| DMR9: 19129001 | 9 | 19129001 | 19130000 | 1000 | 1 | 6.66E−06 | −1.5906467 |
| DMR9: 41235001 | 9 | 41235001 | 41236000 | 1000 | 1 | 5.63E−07 | −1.1919131 |
| DMR9: 41644001 | 9 | 41644001 | 41646000 | 2000 | 1 | 2.98E−06 | −1.8468202 |
| DMR9: 43111001 | 9 | 43111001 | 43112000 | 1000 | 1 | 1.48E−07 | −1.746655 |
| DMR9: 61669001 | 9 | 61669001 | 61670000 | 1000 | 1 | 9.79E−06 | −1.6439159 |
| DMR9: 113083001 | 9 | 113083001 | 113086000 | 3000 | 1 | 1.68E−06 | −3.0084045 |
| DMR9: 125425001 | 9 | 125425001 | 125428000 | 3000 | 1 | 6.86E−06 | −0.8153035 |
| DMR9: 137809001 | 9 | 137809001 | 137811000 | 2000 | 1 | 1.63E−06 | −1.6804834 |
| DMR10: 5512001 | 10 | 5512001 | 5514000 | 2000 | 1 | 4.05E−06 | −2.761433 |
| DMR10: 18917001 | 10 | 18917001 | 18919000 | 2000 | 1 | 1.91E−06 | −1.0126471 |
| DMR10: 29254001 | 10 | 29254001 | 29255000 | 1000 | 1 | 6.67E−06 | 1.2016721 |
| DMR10: 32286001 | 10 | 32286001 | 32287000 | 1000 | 1 | 4.78E−06 | −1.1833967 |
| DMR10: 40846001 | 10 | 40846001 | 40847000 | 1000 | 1 | 2.02E−08 | −1.7704581 |
| DMR10: 45786001 | 10 | 45786001 | 45787000 | 1000 | 1 | 7.22E−06 | −0.7814647 |
| DMR10: 67563001 | 10 | 67563001 | 67564000 | 1000 | 1 | 1.83E−06 | −1.003488 |
| DMR10: 76034001 | 10 | 76034001 | 76036000 | 2000 | 1 | 2.93E−06 | −0.8510863 |
| DMR10: 125192001 | 10 | 125192001 | 125194000 | 2000 | 1 | 2.87E−06 | −0.8868839 |
| DMR10: 125896001 | 10 | 125896001 | 125899000 | 3000 | 1 | 9.30E−06 | −1.9587022 |
| DMR10: 132443001 | 10 | 132443001 | 132445000 | 2000 | 1 | 2.13E−06 | 1.6143491 |
| DMR10: 132858001 | 10 | 132858001 | 132860000 | 2000 | 1 | 4.83E−06 | −1.494898 |
| DMR11: 33429001 | 11 | 33429001 | 33431000 | 2000 | 1 | 4.80E−08 | 0.9745725 |
| DMR11: 64607001 | 11 | 64607001 | 64608000 | 1000 | 1 | 9.76E−06 | −1.5046308 |
| DMR11: 71593001 | 11 | 71593001 | 71594000 | 1000 | 1 | 3.91E−06 | −1.3475282 |
| DMR11: 134288001 | 11 | 134288001 | 134289000 | 1000 | 1 | 9.06E−06 | −1.8607813 |
| DMR12: 1638001 | 12 | 1638001 | 1640000 | 2000 | 1 | 3.50E−06 | −1.9194063 |
| DMR12: 10027001 | 12 | 10027001 | 10028000 | 1000 | 1 | 4.47E−06 | 0.8480909 |
| DMR12: 22036001 | 12 | 22036001 | 22037000 | 1000 | 1 | 5.92E−06 | −1.0274171 |
| DMR12: 35064001 | 12 | 35064001 | 35065000 | 1000 | 1 | 2.24E−06 | −1.5044425 |
| DMR12: 54195001 | 12 | 54195001 | 54197000 | 2000 | 1 | 6.68E−06 | 0.8359955 |
| DMR12: 56500001 | 12 | 56500001 | 56501000 | 1000 | 1 | 9.18E−06 | −0.9025905 |
| DMR12: 56595001 | 12 | 56595001 | 56596000 | 1000 | 1 | 2.90E−06 | −1.3567206 |
| DMR12: 117606001 | 12 | 117606001 | 117607000 | 1000 | 1 | 9.33E−06 | 1.4461211 |
| DMR12: 124208001 | 12 | 124208001 | 124209000 | 1000 | 1 | 9.81E−06 | −2.2382942 |
| DMR12: 131087001 | 12 | 131087001 | 131089000 | 2000 | 1 | 9.95E−06 | −2.0205843 |
| DMR13: 23084001 | 13 | 23084001 | 23086000 | 2000 | 1 | 1.30E−06 | −1.2929502 |
| DMR13: 30392001 | 13 | 30392001 | 30393000 | 1000 | 1 | 4.82E−06 | −0.8768676 |
| DMR13: 57140001 | 13 | 57140001 | 57144000 | 4000 | 1 | 3.02E−06 | −2.431105 |
| DMR13: 57146001 | 13 | 57146001 | 57151000 | 5000 | 3 | 9.83E−07 | −2.3210882 |
| DMR13: 57152001 | 13 | 57152001 | 57157000 | 5000 | 1 | 5.11E−06 | −2.4181847 |
| DMR13: 57165001 | 13 | 57165001 | 57171000 | 6000 | 2 | 3.23E−07 | −2.8351351 |
| DMR13: 57172001 | 13 | 57172001 | 57174000 | 2000 | 1 | 5.17E−06 | −2.5067514 |
| DMR13: 76849001 | 13 | 76849001 | 76850000 | 1000 | 1 | 3.96E−06 | −1.5185848 |
| DMR13: 113834001 | 13 | 113834001 | 113835000 | 1000 | 1 | 1.98E−06 | −1.5471945 |
| DMR14: 19337001 | 14 | 19337001 | 19340000 | 3000 | 1 | 4.91E−06 | −1.7315727 |
| DMR14: 19361001 | 14 | 19361001 | 19363000 | 2000 | 1 | 2.26E−08 | −1.9640587 |
| DMR14: 19678001 | 14 | 19678001 | 19680000 | 2000 | 1 | 3.12E−06 | −2.2198228 |
| DMR14: 70233001 | 14 | 70233001 | 70235000 | 2000 | 1 | 3.03E−06 | −1.956227 |
| DMR15: 20799001 | 15 | 20799001 | 20801000 | 2000 | 1 | 1.10E−06 | −2.2910423 |
| DMR15: 25389001 | 15 | 25389001 | 25391000 | 2000 | 1 | 8.18E−07 | −1.0766537 |
| DMR15: 31442001 | 15 | 31442001 | 31443000 | 1000 | 1 | 7.44E−06 | −1.7221919 |
| DMR15: 47155001 | 15 | 47155001 | 47156000 | 1000 | 1 | 6.14E−08 | −1.5006382 |
| DMR16: 1092001 | 16 | 1092001 | 1096000 | 4000 | 2 | 2.94E−06 | −1.2310444 |
| DMR16: 2750001 | 16 | 2750001 | 2752000 | 2000 | 1 | 2.41E−06 | −1.8140343 |
| DMR16: 13235001 | 16 | 13235001 | 13236000 | 1000 | 1 | 4.52E−07 | −1.5067152 |
| DMR16: 34571001 | 16 | 34571001 | 34577000 | 6000 | 4 | 8.99E−07 | −1.243301 |
| DMR16: 34580001 | 16 | 34580001 | 34602000 | 22000 | 4 | 9.45E−07 | −1.2723349 |
| DMR16: 34603001 | 16 | 34603001 | 34612000 | 9000 | 1 | 1.71E−06 | −1.5120863 |
| DMR16: 34717001 | 16 | 34717001 | 34729000 | 12000 | 1 | 8.96E−06 | −1.543084 |
| DMR16: 34946001 | 16 | 34946001 | 34961000 | 15000 | 1 | 5.80E−06 | −1.4539215 |
| DMR16: 46380001 | 16 | 46380001 | 46423000 | 43000 | 8 | 9.08E−07 | −1.2446121 |
| DMR16: 74985001 | 16 | 74985001 | 74986000 | 1000 | 1 | 2.38E−06 | −1.5455224 |
| DMR16: 81051001 | 16 | 81051001 | 81052000 | 1000 | 1 | 6.78E−06 | −1.0652411 |
| DMR16: 86681001 | 16 | 86681001 | 86684000 | 3000 | 1 | 1.91E−07 | −1.639672 |
| DMR16: 88184001 | 16 | 88184001 | 88186000 | 2000 | 1 | 7.72E−08 | −2.1978318 |
| DMR16: 88531001 | 16 | 88531001 | 88533000 | 2000 | 1 | 8.62E−07 | −2.0612887 |
| DMR16: 89281001 | 16 | 89281001 | 89282000 | 1000 | 1 | 5.15E−06 | −1.5758789 |
| DMR17: 2692001 | 17 | 2692001 | 2693000 | 1000 | 1 | 4.59E−06 | −2.0005593 |
| DMR17: 8227001 | 17 | 8227001 | 8229000 | 2000 | 1 | 3.68E−06 | −1.291337 |
| DMR17: 24421001 | 17 | 24421001 | 24422000 | 1000 | 1 | 6.34E−07 | −1.6340753 |
| DMR17: 25074001 | 17 | 25074001 | 25075000 | 1000 | 1 | 2.20E−06 | −1.8468259 |
| DMR17: 26625001 | 17 | 26625001 | 26627000 | 2000 | 1 | 4.27E−07 | −1.9925314 |
| DMR17: 26881001 | 17 | 26881001 | 26886000 | 5000 | 2 | 5.76E−06 | −1.4743264 |
| DMR17: 31561001 | 17 | 31561001 | 31562000 | 1000 | 1 | 6.61E−07 | −1.2921192 |
| DMR17: 42404001 | 17 | 42404001 | 42406000 | 2000 | 1 | 9.49E−07 | −1.8382718 |
| DMR17: 50220001 | 17 | 50220001 | 50222000 | 2000 | 1 | 1.21E−06 | −1.6834102 |
| DMR17: 80661001 | 17 | 80661001 | 80662000 | 1000 | 1 | 1.66E−06 | −1.0482083 |
| DMR17: 82359001 | 17 | 82359001 | 82362000 | 3000 | 1 | 4.41E−06 | 1.1770702 |
| DMR18: 9868001 | 18 | 9868001 | 9869000 | 1000 | 1 | 6.69E−06 | −0.8901627 |
| DMR18: 12375001 | 18 | 12375001 | 12376000 | 1000 | 1 | 5.16E−06 | −1.8792193 |
| DMR18: 14488001 | 18 | 14488001 | 14490000 | 2000 | 1 | 6.26E−06 | −1.582541 |
| DMR18: 20578001 | 18 | 20578001 | 20580000 | 2000 | 1 | 4.60E−06 | −1.3916313 |
| DMR18: 40983001 | 18 | 40983001 | 40985000 | 2000 | 1 | 4.12E−06 | 0.8892648 |
| DMR18: 76611001 | 18 | 76611001 | 76613000 | 2000 | 2 | 8.41E−07 | −2.0375198 |
| DMR19: 9232001 | 19 | 9232001 | 9234000 | 2000 | 1 | 3.12E−06 | −1.2938746 |
| DMR19: 15456001 | 19 | 15456001 | 15457000 | 1000 | 1 | 4.17E−06 | −1.7724757 |
| DMR19: 29638001 | 19 | 29638001 | 29641000 | 3000 | 1 | 1.08E−06 | −1.6066412 |
| DMR19: 36273001 | 19 | 36273001 | 36310000 | 37000 | 5 | 1.07E−07 | −2.8235712 |
| DMR19: 37269001 | 19 | 37269001 | 37304000 | 35000 | 3 | 1.06E−06 | −2.5754265 |
| DMR19: 39482001 | 19 | 39482001 | 39483000 | 1000 | 1 | 7.68E−06 | −1.0961949 |
| DMR19: 45474001 | 19 | 45474001 | 45475000 | 1000 | 1 | 1.19E−06 | −1.5692376 |
| DMR19: 49209001 | 19 | 49209001 | 49212000 | 3000 | 1 | 5.35E−06 | −1.0071734 |
| DMR19: 49862001 | 19 | 49862001 | 49863000 | 1000 | 1 | 4.63E−07 | −2.1268905 |
| DMR19: 50908001 | 19 | 50908001 | 50909000 | 1000 | 1 | 4.46E−06 | −1.0203746 |
| DMR19: 53768001 | 19 | 53768001 | 53769000 | 1000 | 1 | 9.73E−06 | −1.1968045 |
| DMR19: 55616001 | 19 | 55616001 | 55617000 | 1000 | 1 | 4.94E−06 | −1.6534294 |
| DMR19: 56197001 | 19 | 56197001 | 56198000 | 1000 | 1 | 1.38E−07 | −2.5628136 |
| DMR20: 419001 | 20 | 419001 | 422000 | 3000 | 1 | 8.84E−08 | −2.3117104 |
| DMR20: 5069001 | 20 | 5069001 | 5070000 | 1000 | 1 | 2.76E−06 | 0.8082335 |
| DMR20: 18092001 | 20 | 18092001 | 18094000 | 2000 | 1 | 6.51E−06 | −1.8216619 |
| DMR20: 23365001 | 20 | 23365001 | 23366000 | 1000 | 1 | 2.90E−06 | −1.7760581 |
| DMR20: 26696001 | 20 | 26696001 | 26697000 | 1000 | 1 | 3.59E−06 | −1.9464181 |
| DMR20: 27990001 | 20 | 27990001 | 27991000 | 1000 | 1 | 4.21E−06 | −1.905119 |
| DMR20: 63970001 | 20 | 63970001 | 63971000 | 1000 | 1 | 9.14E−06 | 1.3517273 |
| DMR21: 8806001 | 21 | 8806001 | 8816000 | 10000 | 2 | 1.42E−07 | −1.6852815 |
| DMR21: 9067001 | 21 | 9067001 | 9071000 | 4000 | 2 | 4.72E−06 | −1.527546 |
| DMR21: 9086001 | 21 | 9086001 | 9089000 | 3000 | 1 | 3.73E−06 | −1.1750689 |
| DMR21: 9872001 | 21 | 9872001 | 9874000 | 2000 | 1 | 4.96E−06 | −0.9015005 |
| DMR21: 12679001 | 21 | 12679001 | 12680000 | 1000 | 1 | 3.89E−06 | −1.9111383 |
| DMR21: 20781001 | 21 | 20781001 | 20783000 | 2000 | 1 | 3.21E−06 | −1.8885383 |
| DMR21: 32277001 | 21 | 32277001 | 32278000 | 1000 | 1 | 6.65E−06 | −1.5881648 |
| DMR21: 37919001 | 21 | 37919001 | 37920000 | 1000 | 1 | 4.79E−06 | −1.4685089 |
| DMR21: 46007001 | 21 | 46007001 | 46008000 | 1000 | 1 | 3.56E−06 | −1.0562453 |
| DMR22: 10576001 | 22 | 10576001 | 10577000 | 1000 | 1 | 6.16E−08 | −1.5166733 |
| DMR22: 10703001 | 22 | 10703001 | 10704000 | 1000 | 1 | 4.84E−06 | −1.7196769 |
| DMR22: 10738001 | 22 | 10738001 | 10740000 | 2000 | 1 | 6.36E−06 | −1.0923415 |
| DMR22: 10741001 | 22 | 10741001 | 10745000 | 4000 | 1 | 4.03E−06 | −1.758626 |
| DMR22: 11609001 | 22 | 11609001 | 11612000 | 3000 | 1 | 1.54E−06 | −0.8957715 |
| DMR22: 12167001 | 22 | 12167001 | 12179000 | 12000 | 1 | 1.50E−06 | −1.2108072 |
| DMR22: 15563001 | 22 | 15563001 | 15564000 | 1000 | 1 | 5.86E−06 | −1.8164072 |
| DMR22: 15572001 | 22 | 15572001 | 15574000 | 2000 | 1 | 2.20E−06 | −1.4760505 |
| DMR22: 16305001 | 22 | 16305001 | 16343000 | 38000 | 1 | 8.43E−06 | −1.3536179 |
| DMR22: 18845001 | 22 | 18845001 | 18847000 | 2000 | 1 | 9.41E−07 | −1.7097476 |
| DMR22: 24250001 | 22 | 24250001 | 24252000 | 2000 | 1 | 7.29E−06 | −1.6278471 |
| DMR22: 24454001 | 22 | 24454001 | 24455000 | 1000 | 1 | 1.49E−06 | −0.8764264 |
| DMR22: 34157001 | 22 | 34157001 | 34159000 | 2000 | 1 | 5.97E−07 | −1.2803132 |
| DMR22: 37444001 | 22 | 37444001 | 37446000 | 2000 | 1 | 8.88E−08 | −0.8579501 |
| DMR22: 48286001 | 22 | 48286001 | 48287000 | 1000 | 1 | 5.03E−08 | −1.7535225 |
| DMRMT: 1 | MT | 1 | 16569 | 16569 | 17 | 3.41E−10 | −2.3369179 |
| DMRX: 268001 | X | 268001 | 271000 | 3000 | 1 | 9.96E−07 | −1.7180105 |
| DMRX: 10094001 | X | 10094001 | 10095000 | 1000 | 1 | 3.07E−07 | 1.0653938 |
| DMRX: 49339001 | X | 49339001 | 49342000 | 3000 | 1 | 9.07E−06 | −1.178808 |
| DMRX: 49589001 | X | 49589001 | 49591000 | 2000 | 1 | 3.70E−06 | −2.2773116 |
| DMRX: 56594001 | X | 56594001 | 56595000 | 1000 | 1 | 1.85E−06 | −1.2924059 |
| DMRX: 140977001 | X | 140977001 | 140978000 | 1000 | 1 | 5.52E−06 | 0.9405132 |
| DMRY: 6246001 | Y | 6246001 | 6247000 | 1000 | 1 | 1.67E−07 | −2.27506 |
| DMRY: 6265001 | Y | 6265001 | 6266000 | 1000 | 1 | 6.09E−06 | −2.3232095 |
| DMRY: 9356001 | Y | 9356001 | 9357000 | 1000 | 1 | 3.09E−06 | −2.5246157 |
| DMRY: 9395001 | Y | 9395001 | 9400000 | 5000 | 1 | 6.16E−06 | −2.3246137 |
| DMRY: 9505001 | Y | 9505001 | 9509000 | 4000 | 1 | 7.26E−07 | −2.3500189 |
| DMRY: 21896001 | Y | 21896001 | 21897000 | 1000 | 1 | 6.37E−06 | −2.1121986 |

| | CpG | CpG | Gene | Gene |
|---|---|---|---|---|
| DMR Name | # | Density | Annotation | Category |
| DMR1: 629001 | 159 | 2.65 | AL669831.3; MTND1P23; | |
| | | | MTND2P28; MTCO1P12; | |
| | | | AC114498.2; MTCO2P12; | |
| | | | MTATP8P1; MTATP6P1; | |
| | | | MTCO3P12 | |
| DMR1: 4712001 | 55 | 5.5 | AJAP1 | |
| DMR1: 24099001 | 64 | 3.2 | MYOM3 | Cytoskeleton |
| DMR1: 121860001 | 18 | 1.8 | | |
| DMR1: 144475001 | 24 | 2.4 | AC246785.1 | |
| DMR1: 153464001 | 14 | 1.4 | S100A7 | Signaling |
| DMR1: 156732001 | 10 | 1 | ISG20L2; RRNAD1; | Transcription; |
| | | | MRPL24; HDGF | Signaling |
| DMR1: 204834001 | 10 | 1 | NFASC | Extracellular |
| | | | | Matrix |
| DMR1: 226959001 | 7 | 0.7 | COQ8A; AL353689.1 | |
| DMR2: 3591001 | 74 | 7.4 | COLEC11 | Immune |
| DMR2: 10913001 | 83 | 4.15 | KCNF1 | Metabolism |
| DMR2: 87091001 | 7 | 0.7 | | |
| DMR2: 87348001 | 162 | 8.1 | IGKV3OR2-268 | |
| DMR2: 87405001 | 237 | 0.878 | LINC01943 | |
| DMR2: 101342001 | 79 | 3.95 | CREG2 | |
| DMR2: 104368001 | 58 | 2.9 | | |
| DMR2: 131646001 | 47 | 4.7 | LINC01087; GRAMD4P8 | |
| DMR2: 201858001 | 12 | 1.2 | CDK15 | Signaling |
| DMR2: 211358001 | 4 | 0.4 | | |
| DMR2: 225582001 | 71 | 7.1 | NYAP2 | |
| DMR3: 112966001 | 6 | 0.3 | CD200R1 | Receptor |
| DMR3: 125912001 | 33 | 1.65 | LINC02614; ENPP7P4; | |
| | | | AC092903.2; FAM86JP | |
| DMR3: 150778001 | 27 | 2.7 | | |
| DMR3: 176844001 | 9 | 0.9 | LINC01208 | |
| DMR3: 184170001 | 69 | 6.9 | DVL3; AP2M1 | Signaling |
| DMR3: 188433001 | 7 | 0.7 | LPP | Cytoskeleton |
| DMR4: 53001 | 136 | 3.4 | BNIP3P41; ZNF595 | Transcription |
| DMR4: 747001 | 122 | 6.1 | PCGF3; AC139887.4 | Transcription |
| DMR4: 3809001 | 70 | 3.5 | | |
| DMR4: 49270001 | 100 | 2.5 | | |
| DMR4: 49275001 | 132 | 4.4 | | |
| DMR4: 49279001 | 113 | 2.26 | | |
| DMR4: 49291001 | 279 | 2.325 | | |
| DMR4: 49313001 | 107 | 2.14 | | |
| DMR4: 49319001 | 132 | 2.2 | | |
| DMR4: 49510001 | 291 | 2.91 | ANKRD20A17P; AC119751.5; | |
| | | | AC119751.2; AC119751.8 | |
| DMR4: 68471001 | 0 | 0 | TMPRSS11E | Protease |
| DMR4: 69599001 | 3 | 0.3 | UGT2A1; UGT2A2 | Metabolism |
| DMR4: 185384001 | 10 | 1 | LRP2BP; AC112722.1 | Signaling |
| DMR4: 186889001 | 58 | 5.8 | AC108865.1; AC108865.2 | |
| DMR4: 190021001 | 169 | 8.45 | RNA5SP174; RNA5SP175; | |
| | | | DUX4L9; FRG2 | |
| DMR5: 258001 | 343 | 8.575 | SDHA; AC021087.5; | Metabolism; |
| | | | AC021087.1; PDCD6; AHRR | Transcription |
| DMR5: 1570001 | 47 | 4.7 | SDHAP3 | |
| DMR5: 12433001 | 3 | 0.3 | | |
| DMR5: 17581001 | 235 | 3.357 | TAF11L7; AC233724.7; | |
| | | | TAF11L8; TAF11L9; | |
| | | | TAF11L10 | |
| DMR5: 17589001 | 365 | 3.318 | TAF11L7; AC233724.7; | |
| | | | TAF11L8; TAF11L9; | |
| | | | TAF11L10; AC233724.3; | |
| | | | AC233724.6; TAF11L11 | |
| DMR5: 36783001 | 35 | 3.5 | | |
| DMR5: 46622001 | 10 | 1 | | |
| DMR6: 105831001 | 6 | 0.6 | AL591518.1 | |
| DMR6: 132600001 | 4 | 0.2 | TAAR4P; TAAR3P | |
| DMR6: 150337001 | 15 | 1.5 | | |
| DMR6: 163194001 | 64 | 2.133 | PACRG; PACRG-AS3 | Development |
| DMR6: 167731001 | 37 | 3.7 | | |
| DMR6: 170138001 | 43 | 4.3 | | |
| DMR7: 636001 | 119 | 5.95 | PRKAR1B | Signaling |
| DMR7: 2577001 | 196 | 9.8 | IQCE | |
| DMR7: 10779001 | 38 | 3.8 | AC004949.1 | |
| DMR7: 37256001 | 6 | 0.6 | ELMO1 | Signaling |
| DMR7: 58104001 | 513 | 3.206 | | |
| DMR7: 64407001 | 31 | 3.1 | | |
| DMR7: 85091001 | 7 | 0.7 | SEMA3D | Growth Factors |
| | | | | & Cytokines |
| DMR7: 128586001 | 7 | 0.7 | AC090114.3; AC108010.1 | |
| DMR7: 155788001 | 105 | 5.25 | RBM33; SHH | Signaling |
| DMR8: 12541001 | 18 | 1.8 | AC068587.4 | |
| DMR8: 23248001 | 19 | 1.9 | CHMP7 | Binding |
| | | | | Protein |
| DMR8: 42053001 | 31 | 3.1 | KAT6A; RF01169 | Transcription |
| DMR8: 46103001 | 102 | 3.4 | | |
| DMR8: 46257001 | 111 | 2.775 | | |
| DMR8: 52388001 | 30 | 3 | ST18 | Transcription |
| DMR8: 85642001 | 118 | 5.9 | REXO1L8P | |
| DMR8: 85645001 | 566 | 2.979 | REXO1L8P; REXO1L3P; | |
| | | | REXO1L1P | |
| DMR8: 85714001 | 1357 | 2.61 | REXO1L12P; REXO1L11P; | |
| | | | REXO1L10P; REXO1L9P; | |
| | | | REXO1L2P | |
| DMR8: 85767001 | 361 | 2.579 | REXO1L9P; REXO1L2P; | |
| | | | AC232323.1 | |
| DMR8: 85782001 | 1277 | 2.66 | REXO1L2P; AC232323.1; | |
| | | | REXO1L4P; REXO1L5P; | |
| | | | REXO1L6P; AC100801.1 | |
| DMR8: 112511001 | 23 | 2.3 | CSMD3 | |
| DMR8: 133772001 | 36 | 1.8 | AC133634.1; AC090821.1 | |
| DMR9: 3536001 | 3 | 0.3 | RFX3; RFX3-AS1 | Transcription |
| DMR9: 19129001 | 25 | 2.5 | PLIN2 | |
| DMR9: 41235001 | 57 | 5.7 | MIR4477A; RNA5SP530 | |
| DMR9: 41644001 | 79 | 3.95 | AL591926.6; AL591926.5; | |
| | | | AL591926.2 | |
| DMR9: 43111001 | 29 | 2.9 | FP325317.1 | |
| DMR9: 61669001 | 40 | 4 | AL935212.1; AL935212.2 | |
| DMR9: 113083001 | 156 | 5.2 | AL449105.4; AL449105.2; | |
| | | | AL449105.5 | |
| DMR9: 125425001 | 88 | 2.933 | RF00017; MAPKAP1 | Signaling |
| DMR9: 137809001 | 112 | 5.6 | EHMT1 | Transcription |
| DMR10: 5512001 | 64 | 3.2 | CALML3-AS1 | |
| DMR10: 18917001 | 36 | 1.8 | | |
| DMR10: 29254001 | 9 | 0.9 | | |
| DMR10: 32286001 | 12 | 1.2 | EPC1; AL158834.1 | Metabolism |
| DMR10: 40846001 | 15 | 1.5 | | |
| DMR10: 45786001 | 18 | 1.8 | WASHC2C | |
| DMR10: 67563001 | 4 | 0.4 | CTNNA3 | Cytoskeleton |
| DMR10: 76034001 | 46 | 2.3 | LRMDA | |
| DMR10: 125192001 | 47 | 2.35 | | |
| DMR10: 125896001 | 160 | 5.333 | DHX32; RNU2-42P; FANK1 | Transcription |
| DMR10: 132443001 | 44 | 2.2 | AL451069.3; C10orf91 | |
| DMR10: 132858001 | 85 | 4.25 | CFAP46 | |
| DMR11: 33429001 | 24 | 1.2 | KIAA1549L | |
| DMR11: 64607001 | 73 | 7.3 | SLC22A12; NRXN2 | Transport; |
| | | | | Receptor |
| DMR11: 71593001 | 19 | 1.9 | KRTAP5-11; OR7E87P; | |
| | | | UNC93B6 | |
| DMR11: 134288001 | 22 | 2.2 | GLB1L3 | Golgi |
| DMR12: 1638001 | 97 | 4.85 | WNT5B | Signaling |
| DMR12: 10027001 | 5 | 0.5 | CLEC12B; AC024224.2; | |
| | | | CLEC9A | |
| DMR12: 22036001 | 21 | 2.1 | CMAS | Metabolism |
| DMR12: 35064001 | 17 | 1.7 | | |
| DMR12: 54195001 | 28 | 1.4 | SMUG1 | Transcription |
| DMR12: 56500001 | 30 | 3 | | |
| DMR12: 56595001 | 10 | 1 | RBMS2; RNU6-343P; | Translation; |
| | | | BAZ2A | Transcription |
| DMR12: 117606001 | 4 | 0.4 | KSR2 | Signaling |
| DMR12: 124208001 | 27 | 2.7 | RFLNA; AC026358.1 | |
| DMR12: 131087001 | 85 | 4.25 | ADGRD1 | |
| DMR13: 23084001 | 50 | 2.5 | | |
| DMR13: 30392001 | 14 | 1.4 | AL161893.1 | |
| DMR13: 57140001 | 144 | 3.6 | PRR20A; PRR20C; | |
| | | | PRR20B | |
| DMR13: 57146001 | 183 | 3.66 | PRR20A; PRR20C; | |
| | | | PRR20B; PRR20D | |
| DMR13: 57152001 | 184 | 3.68 | PRR20A; PRR20C; | |
| | | | PRR20B; PRR20D | |
| DMR13: 57165001 | 204 | 3.4 | PRR20C; PRR20D; | |
| | | | PRR20E; PRR20FP | |
| DMR13: 57172001 | 80 | 4 | PRR20D; PRR20E; | |
| | | | PRR20FP | |
| DMR13: 76849001 | 19 | 1.9 | AL136441.1; AL365394.1 | |
| DMR13: 113834001 | 52 | 5.2 | GAS6-AS1; GAS6 | Signaling |
| DMR14: 19337001 | 80 | 2.667 | AL589743.1; LINC01297 | |
| DMR14: 19361001 | 106 | 5.3 | LINC01297; GRAMD4P3 | |
| DMR14: 19678001 | 56 | 2.8 | AL512310.11; AL512310.4; | |
| | | | AL512310.5; AL512310.6; | |
| | | | AL512310.9; AL512310.7; | |
| | | | AL512310.2; ARHGAP42P4 | |
| DMR14: 70233001 | 70 | 3.5 | AL160191.1; AL160191.3; | |
| | | | AL160191.2 | |
| DMR15: 20799001 | 104 | 5.2 | AC012414.7 | |
| DMR15: 25389001 | 14 | 0.7 | SNHG14; UBE3A | Metabolism |
| DMR15: 31442001 | 21 | 2.1 | KLF13 | Transcription |
| DMR15: 47155001 | 22 | 2.2 | | |
| DMR16: 1092001 | 216 | 5.4 | C1QTNF8; AL031713.1 | Immune |
| DMR16: 2750001 | 59 | 2.95 | SRRM2-AS1; SRRM2 | |
| DMR16: 13235001 | 32 | 3.2 | SHISA9 | |
| DMR16: 34571001 | 200 | 3.333 | | |
| DMR16: 34580001 | 759 | 3.45 | | |
| DMR16: 34603001 | 341 | 3.789 | | |
| DMR16: 34717001 | 432 | 3.6 | | |
| DMR16: 34946001 | 547 | 3.647 | AC135776.4 | |
| DMR16: 46380001 | 1611 | 3.747 | | |
| DMR16: 74985001 | 61 | 6.1 | WDR59 | |
| DMR16: 81051001 | 7 | 0.7 | AC092718.8; ATMIN; C16orf46; | DNA Repair |
| | | | AC092718.3; AC092718.5 | |
| DMR16: 86681001 | 85 | 2.833 | | |
| DMR16: 88184001 | 77 | 3.85 | AC134312.2; AC134312.5; | |
| | | | AC134312.6; LINC02182 | |
| DMR16: 88531001 | 83 | 4.15 | ZFPM1; AC116552.1 | Transcription |
| DMR16: 89281001 | 66 | 6.6 | ANKRD11; AC137932.3 | EST |
| DMR17: 2692001 | 76 | 7.6 | PAFAH1B1; AC005696.2; | Metabolism |
| | | | AC005696.3; CLUH; MIR6776 | |
| DMR17: 8227001 | 38 | 1.9 | LINC00324; CTC1 | |
| DMR17: 24421001 | 19 | 1.9 | | |
| DMR17: 25074001 | 26 | 2.6 | | |
| DMR17: 26625001 | 34 | 1.7 | | |
| DMR17: 26881001 | 68 | 1.36 | | |
| DMR17: 31561001 | 13 | 1.3 | MIR193A; AC003101.2; | |
| | | | RNU6ATAC7P; AC003101.1 | |
| DMR17: 42404001 | 141 | 7.05 | CAVIN1 | |
| DMR17: 50220001 | 44 | 2.2 | AC015909.1; AC015909.4 | |
| DMR17: 80661001 | 33 | 3.3 | RPTOR | |
| DMR17: 82359001 | 10 | 0.333 | TEX19; AC132938.4 | |
| DMR18: 9868001 | 21 | 2.1 | RAB31 | Signaling |
| DMR18: 12375001 | 43 | 4.3 | AFG3L2 | Protease |
| DMR18: 14488001 | 92 | 4.6 | CXADRP3; GRAMD4P7 | |
| DMR18: 20578001 | 38 | 1.9 | | |
| DMR18: 40983001 | 9 | 0.45 | | |
| DMR18: 76611001 | 85 | 4.25 | LINC00683; AC034110.1 | |
| DMR19: 9232001 | 43 | 2.15 | OR7D1P | |
| DMR19: 15456001 | 31 | 3.1 | WIZ; MIR1470; RASAL3 | |
| DMR19: 29638001 | 78 | 2.6 | | |
| DMR19: 36273001 | 2586 | 6.989 | AC012617.1; LINC00665 | |
| DMR19: 37269001 | 2445 | 6.986 | AC016590.1; LINCO1535; | Transcription |
| | | | HKR1 | |
| DMR19: 39482001 | 27 | 2.7 | SUPT5H; TIMM50 | Transcription; |
| | | | | Metabolism |
| DMR19: 45474001 | 37 | 3.7 | ERCC1; FOSB | Epigenetic; |
| | | | | Transcription |
| DMR19: 49209001 | 94 | 3.133 | TRPM4 | Development |
| DMR19: 49862001 | 63 | 6.3 | PTOV1; AC018766.1; | Development; |
| | | | MIR4749; PTOV1-AS2; | DNA Repair |
| | | | PNKP; AKT1S1 | |
| DMR19: 50908001 | 36 | 3.6 | KLK4 | Protease |
| DMR19: 53768001 | 52 | 5.2 | MIR1283-2; RNU6-1041P; | |
| | | | MIR516A2; AC011453.1; | |
| | | | MIR519A2; RNU6-165P; | |
| | | | HMGN1P32; SEPT7P8; | |
| | | | AC008753.1 | |
| DMR19: 55616001 | 90 | 9 | ZNF865; AC008735.4; | Transcription |
| | | | ZNF784 | |
| DMR19: 56197001 | 50 | 5 | ZSCAN5B; ZSCAN5C | Transcription |
| DMR20: 419001 | 121 | 4.033 | RBCK1 | Metabolism |
| DMR20: 5069001 | 11 | 1.1 | AL121890.5; AL121890.4; | |
| | | | TMEM230 | |
| DMR20: 18092001 | 59 | 2.95 | RPL15P1; RNU7-137P | |
| DMR20: 23365001 | 67 | 6.7 | LINC01431; GZF1; NAPB | Transcription |
| DMR20: 26696001 | 18 | 1.8 | | |
| DMR20: 27990001 | 17 | 1.7 | | |
| DMR20: 63970001 | 46 | 4.6 | ZNF512B; SAMD10 | Transcription |
| DMR21: 8806001 | 317 | 3.17 | CR381670.2; SNX18P10 | |
| DMR21: 9067001 | 110 | 2.75 | CR392039.5; TEKT4P2 | |
| DMR21: 9086001 | 125 | 4.167 | TEKT4P2; CR392039.4; | |
| | | | CR392039.1 | |
| DMR21: 9872001 | 18 | 0.9 | | |
| DMR21: 12679001 | 18 | 1.8 | | |
| DMR21: 20781001 | 39 | 1.95 | LINC00320 | |
| DMR21: 32277001 | 28 | 2.8 | MIS18A; MIS18A-AS1 | |
| DMR21: 37919001 | 12 | 1.2 | KCNJ6 | Metabolism |
| DMR21: 46007001 | 25 | 2.5 | COL6A1 | Cytoskeleton |
| DMR22: 10576001 | 10 | 1 | | |
| DMR22: 10703001 | 6 | 0.6 | | |
| DMR22: 10738001 | 32 | 1.6 | RF00004 | |
| DMR22: 10741001 | 162 | 4.05 | RF00004 | |
| DMR22: 11609001 | 77 | 2.567 | | |
| DMR22: 12167001 | 342 | 2.85 | | |
| DMR22: 15563001 | 26 | 2.6 | AP000534.2; ARHGAP42P3; | |
| | | | AP000534.1; AP000533.2; | |
| | | | AP000533.1 | |
| DMR22: 15572001 | 79 | 3.95 | AP000534.2; AP000533.2; | |
| | | | AP000533.1 | |
| DMR22: 16305001 | 1370 | 3.605 | | |
| DMR22: 18845001 | 87 | 4.35 | GGT3P; AC008132.1; | |
| | | | BCRP7 | |
| DMR22: 24250001 | 82 | 4.1 | GGT5; GGTLC4P; | Metabolism |
| | | | POM121L9P; BCRP1 | |
| DMR22: 24454001 | 13 | 1.3 | ADORA2A-AS1 | |
| DMR22: 34157001 | 41 | 2.05 | LINC01643 | |
| DMR22: 37444001 | 41 | 2.05 | | |
| DMR22: 48286001 | 15 | 1.5 | | |
| DMRMT: 1 | 435 | 2.625 | MT-TF; MT-RNR1; MT-TV; | |
| | | | MT-RNR2; MT-TL1; MT-ND1; | |
| | | | MT-TI; MT-TQ; MT-TM; | |
| | | | MT-ND2; MT-TW; MT-TA; | |
| | | | MT-TN; MT-TC; MT-TY; | |
| | | | MT-CO1; MT-TS1; MT-TD; | |
| | | | MT-CO2; MT-TK; MT-ATP8; | |
| | | | MT-ATP6; MT-CO3; MT-TG; | |
| | | | MT-ND3; MT-TR; MT-ND4L; | |
| | | | MT-ND4; MT-TH; MT-TS2; | |
| | | | MT-TL2; MT-ND5; MT-ND6; | |
| | | | MT-TE; MT-CYB; MT-TT; | |
| | | | MT-TP | |
| DMRX: 268001 | 136 | 4.533 | PLCXD1 | |
| DMRX: 10094001 | 17 | 1.7 | WWC3 | |
| DMRX: 49339001 | 113 | 3.767 | GAGE12J; GAGE13; | |
| | | | GAGE2E | |
| DMRX: 49589001 | 87 | 4.35 | GAGE12H; GAGE1; | |
| | | | GAGE2A | |
| DMRX: 56594001 | 10 | 1 | | |
| DMRX: 140977001 | 9 | 0.9 | AL451048.1 | |
| DMRY: 6246001 | 64 | 6.4 | TTTY23B; TSPY2; | Protein |
| | | | FAM197Y9 | Binding |
| DMRY: 6265001 | 55 | 5.5 | FAM197Y9; TSPY11P; | |
| | | | AC006335.2 | |
| DMRY: 9356001 | 59 | 5.9 | FAM197Y8; TSPY8 | Protein |
| | | | | Binding |
| DMRY: 9395001 | 204 | 4.08 | FAM197Y6; AC006158.3; | Protein |
| | | | TSPY3 | Binding |
| DMRY: 9505001 | 188 | 4.7 | FAM197Y3; TSPY6P; | |
| | | | FAM197Y2 | |
| DMRY: 21896001 | 30 | 3 | RBMY1D; AC007322.4; | Transcription |
| | | | RBMY1E | |

In some embodiments, the subject may be infertile. In some embodiments, the subject may have a reduced fertility compared to a normal subject that has no fertility issues. In some embodiments, the present disclosure provides administering a treatment to the subject that may be infertile or is at risk of being infertile. In some embodiments, the present disclosure provides administering a treatment to the subject that may have reduced fertility compared to a normal subject. In some embodiments, the treatment may be any hormone therapies that may increase sperm number and motility. The hormone therapy may be administering a therapeutically effective amount of follicle stimulating hormone (FSH), or an analog thereof to a subject. In some embodiments, the hormone therapy may be administering a therapeutically effective amount of human menopausal gonadotropin (hMG), or an analog thereof to the subject. In some embodiments, the treatment may comprise performing in vitro fertilization (IVF), intracytoplasmic sperm injection (ICSI), or any other suitable procedures that may result in successful pregnancy.

Further, the present disclosure provides methods for detecting a methylation alteration of a portion of said nucleic acid sequence comprised in a differential DNA methylation region (DMR) listed in Table 3, thereby generating an epigenetic profile; and analyzing said epigenetic profile using a computer processor by comparing to a reference epigenetic profile of methylation status of at least a portion of corresponding nucleic acid sequence comprised in said DMR listed in Table 3. For example, if a DMR from Table 3 comprises 1000 bp nucleotides, in some embodiments, at least about 50%, about 60%, about 70%, about 80%, about 90%, or about 100% of the 1000 bp nucleotides may be measured to determine the methylation level.

TABLE 3
Responder versus non-responder DMRs with various genomic features

| | | | | | # Sig | | |
|---|---|---|---|---|---|---|---|
| DMR Name | Chr | Start | Stop | Length | Win | minP | maxLFC |
| DMR1: 17235001 | 1 | 17235001 | 17236000 | 1000 | 1 | 9.11E−07 | 1.0645622 |
| DMR1: 25292001 | 1 | 25292001 | 25294000 | 2000 | 1 | 9.84E−09 | −2.1663744 |
| DMR1: 25299001 | 1 | 25299001 | 25300000 | 1000 | 1 | 2.76E−09 | −1.8483392 |
| DMR1: 25304001 | 1 | 25304001 | 25305000 | 1000 | 1 | 8.29E−06 | −1.8151307 |
| DMR1: 25318001 | 1 | 25318001 | 25319000 | 1000 | 1 | 1.70E−07 | −2.0585241 |
| DMR1: 25333001 | 1 | 25333001 | 25334000 | 1000 | 1 | 2.49E−06 | −1.3360508 |
| DMR1: 218548001 | 1 | 218548001 | 218549000 | 1000 | 1 | 7.63E−06 | −1.2569819 |
| DMR2: 163904001 | 2 | 163904001 | 163905000 | 1000 | 1 | 6.54E−06 | −1.4222477 |
| DMR3: 9159001 | 3 | 9159001 | 9160000 | 1000 | 1 | 7.15E−06 | −1.0760625 |
| DMR3: 18212001 | 3 | 18212001 | 18213000 | 1000 | 1 | 1.06E−06 | −1.4381627 |
| DMR3: 25359001 | 3 | 25359001 | 25360000 | 1000 | 1 | 8.09E−06 | −1.2918398 |
| DMR3: 36185001 | 3 | 36185001 | 36186000 | 1000 | 1 | 1.81E−07 | −1.3766023 |
| DMR3: 111089001 | 3 | 111089001 | 111090000 | 1000 | 1 | 8.67E−07 | −1.5806562 |
| DMR3: 118291001 | 3 | 118291001 | 118293000 | 2000 | 1 | 4.57E−08 | −1.4412971 |
| DMR3: 120974001 | 3 | 120974001 | 120977000 | 3000 | 1 | 5.90E−06 | −1.2434688 |
| DMR4: 164482001 | 4 | 164482001 | 164483000 | 1000 | 1 | 2.00E−06 | 1.3078442 |
| DMR4: 170150001 | 4 | 170150001 | 170151000 | 1000 | 1 | 4.27E−07 | −1.5550518 |
| DMR4: 187047001 | 4 | 187047001 | 187048000 | 1000 | 1 | 8.29E−06 | 1.1028096 |
| DMR5: 15460001 | 5 | 15460001 | 15461000 | 1000 | 1 | 5.64E−06 | −0.9900734 |
| DMR5: 20858001 | 5 | 20858001 | 20859000 | 1000 | 1 | 8.86E−06 | −1.1168998 |
| DMR5: 84058001 | 5 | 84058001 | 84059000 | 1000 | 1 | 3.43E−06 | −1.3418608 |
| DMR5: 113458001 | 5 | 113458001 | 113460000 | 2000 | 1 | 6.47E−06 | 1.1312994 |
| DMR5: 122270001 | 5 | 122270001 | 122271000 | 1000 | 1 | 8.74E−07 | −1.3435475 |
| DMR6: 5284001 | 6 | 5284001 | 5285000 | 1000 | 1 | 2.64E−06 | −1.2871156 |
| DMR6: 169552001 | 6 | 169552001 | 169554000 | 2000 | 1 | 3.18E−06 | 1.5327536 |
| DMR7: 69279001 | 7 | 69279001 | 69280000 | 1000 | 1 | 4.90E−06 | 1.0949466 |
| DMR7: 119280001 | 7 | 119280001 | 119281000 | 1000 | 1 | 3.05E−07 | −1.1363468 |
| DMR8: 10912001 | 8 | 10912001 | 10913000 | 1000 | 1 | 1.65E−06 | −1.6874264 |
| DMR8: 55562001 | 8 | 55562001 | 55563000 | 1000 | 1 | 7.25E−06 | −1.2043517 |
| DMR9: 17319001 | 9 | 17319001 | 17320000 | 1000 | 1 | 9.15E−06 | −1.3272344 |
| DMR9: 22122001 | 9 | 22122001 | 22123000 | 1000 | 1 | 1.03E−07 | −1.5186233 |
| DMR9: 65745001 | 9 | 65745001 | 65746000 | 1000 | 1 | 6.00E−06 | −1.2615133 |
| DMR9: 89370001 | 9 | 89370001 | 89374000 | 4000 | 1 | 9.89E−06 | 1.2449168 |
| DMR9: 124203001 | 9 | 124203001 | 124204000 | 1000 | 1 | 2.36E−06 | 1.1490304 |
| DMR9: 130473001 | 9 | 130473001 | 130474000 | 1000 | 1 | 1.19E−06 | 1.6118581 |
| DMR9: 134476001 | 9 | 134476001 | 134477000 | 1000 | 1 | 6.96E−08 | −1.300523 |
| DMR10: 559001 | 10 | 559001 | 560000 | 1000 | 1 | 4.86E−06 | 1.0297783 |
| DMR10: 3718001 | 10 | 3718001 | 3719000 | 1000 | 1 | 1.33E−06 | 1.5500791 |
| DMR10: 7136001 | 10 | 7136001 | 7137000 | 1000 | 1 | 9.63E−07 | −1.6184753 |
| DMR10: 36569001 | 10 | 36569001 | 36570000 | 1000 | 1 | 1.59E−07 | −1.4504064 |
| DMR11: 19552001 | 11 | 19552001 | 19553000 | 1000 | 1 | 1.12E−06 | 1.327054 |
| DMR11: 126188001 | 11 | 126188001 | 126189000 | 1000 | 1 | 1.92E−06 | 0.991244 |
| DMR12: 62236001 | 12 | 62236001 | 62238000 | 2000 | 1 | 6.81E−06 | −1.1166275 |
| DMR12: 121750001 | 12 | 121750001 | 121752000 | 2000 | 1 | 9.27E−06 | 1.3819881 |
| DMR12: 131647001 | 12 | 131647001 | 131648000 | 1000 | 1 | 7.17E−06 | 1.084876 |
| DMR13: 37820001 | 13 | 37820001 | 37821000 | 1000 | 1 | 8.53E−08 | −1.8339284 |
| DMR14: 81859001 | 14 | 81859001 | 81860000 | 1000 | 1 | 1.86E−06 | −1.3402382 |
| DMR16: 23408001 | 16 | 23408001 | 23409000 | 1000 | 1 | 2.13E−06 | 1.2552037 |
| DMR16: 28375001 | 16 | 28375001 | 28376000 | 1000 | 1 | 1.44E−06 | 1.3194317 |
| DMR17: 45915001 | 17 | 45915001 | 45916000 | 1000 | 1 | 8.73E−06 | 0.8078421 |
| DMR17: 46235001 | 17 | 46235001 | 46236000 | 1000 | 1 | 4.74E−06 | 1.0572367 |
| DMR17: 46297001 | 17 | 46297001 | 46298000 | 1000 | 1 | 5.15E−06 | 0.9689937 |
| DMR17: 73426001 | 17 | 73426001 | 73428000 | 2000 | 1 | 4.27E−06 | −1.3244611 |
| DMR19: 2712001 | 19 | 2712001 | 2715000 | 3000 | 1 | 3.78E−06 | 1.1579408 |
| DMR20: 29885001 | 20 | 29885001 | 29886000 | 1000 | 1 | 2.32E−06 | 1.3647393 |
| DMRX: 119779001 | X | 119779001 | 119780000 | 1000 | 1 | 7.18E−06 | 1.161398 |

| | CpG | CpG | | Gene |
|---|---|---|---|---|
| DMR Name | # | Density | Gene Annotation | Category |
| DMR1: 17235001 | 12 | 1.2 | PADI1 | Metabolism |
| DMR1: 25292001 | 11 | 0.55 | RSRP1; RHD; SDHDP6 | Transport |
| DMR1: 25299001 | 23 | 2.3 | RSRP1; RHD; SDHDP6 | Transport |
| DMR1: 25304001 | 19 | 1.9 | RSRP1; RHD; SDHDP6 | Transport |
| DMR1: 25318001 | 15 | 1.5 | RSRP1; RHD | Transport |
| DMR1: 25333001 | 16 | 1.6 | RSRP1; RHD; AL928711.1; | Transport |
| | | | TMEM50A | |
| DMR1: 218548001 | 13 | 1.3 | RF00012 | |
| DMR2: 163904001 | 4 | 0.4 | AC016766.1 | |
| DMR3: 9159001 | 13 | 1.3 | SRGAP3 | Signaling |
| DMR3: 18212001 | 5 | 0.5 | TBC1D5 | Signaling |
| DMR3: 25359001 | 3 | 0.3 | RARB; RNA5SP126 | Signaling |
| DMR3: 36185001 | 5 | 0.5 | | |
| DMR3: 111089001 | 8 | 0.8 | NECTIN3 | |
| DMR3: 118291001 | 12 | 0.6 | AC068633.1 | |
| DMR3: 120974001 | 20 | 0.667 | STXBP5L | Transcription |
| DMR4: 164482001 | 4 | 0.4 | | |
| DMR4: 170150001 | 4 | 0.4 | AC069306.1 | |
| DMR4: 187047001 | 16 | 1.6 | AC110772.2 | |
| DMR5: 15460001 | 6 | 0.6 | AC114964.1 | |
| DMR5: 20858001 | 7 | 0.7 | LINC02241 | |
| DMR5: 84058001 | 7 | 0.7 | EDIL3 | Extracellular |
| | | | | Matrix |
| DMR5: 113458001 | 31 | 1.55 | MCC | Transcription |
| DMR5: 122270001 | 4 | 0.4 | | |
| DMR6: 5284001 | 11 | 1.1 | FARS2; AL121978.1 | |
| DMR6: 169552001 | 42 | 2.1 | AL031315.1; WDR27 | |
| DMR7: 69279001 | 11 | 1.1 | AC092100.1 | |
| DMR7: 119280001 | 5 | 0.5 | | |
| DMR8: 10912001 | 7 | 0.7 | XKR6 | Immune |
| DMR8: 55562001 | 4 | 0.4 | | |
| DMR9: 17319001 | 5 | 0.5 | CNTLN | |
| DMR9: 22122001 | 5 | 0.5 | CDKN2B-AS1; | |
| | | | RF01909 | |
| DMR9: 65745001 | 7 | 0.7 | FOXD4L4 | |
| DMR9: 89370001 | 56 | 1.4 | SEMA4D | Development |
| DMR9: 124203001 | 18 | 1.8 | | |
| DMR9: 130473001 | 23 | 2.3 | ASS1 | Development |
| DMR9: 134476001 | 12 | 1.2 | | |
| DMR10: 559001 | 21 | 2.1 | DIP2C | |
| DMR10: 3718001 | 16 | 1.6 | | |
| DMR10: 7136001 | 14 | 1.4 | | |
| DMR10: 36569001 | 20 | 2 | | |
| DMR11: 19552001 | 18 | 1.8 | NAV2 | Development |
| DMR11: 126188001 | 21 | 2.1 | AP001893.2 | |
| DMR12: 62236001 | 22 | 1.1 | FAM19A2; KLF17P1 | Growth Factors |
| | | | | & Cytokines |
| DMR12: 121750001 | 18 | 0.9 | TMEM120B | Unknown |
| DMR12: 131647001 | 23 | 2.3 | AC117500.4; LINC02414 | |
| DMR13: 37820001 | 4 | 0.4 | TRPC4 | Development |
| DMR14: 81859001 | 14 | 1.4 | AL355838.1 | |
| DMR16: 23408001 | 34 | 3.4 | COG7; RN7SKP23 | Golgi |
| DMR16: 28375001 | 13 | 1.3 | AC138894.3; EIF3CL | Translation |
| DMR17: 45915001 | 12 | 1.2 | MAPT; CR936218.2 | Cytoskeleton |
| DMR17: 46235001 | 13 | 1.3 | KANSL1; MAPK8IP1P1 | |
| DMR17: 46297001 | 11 | 1.1 | ARL17B; LRRC37A | Signaling |
| DMR17: 73426001 | 24 | 1.2 | SDK2 | Development |
| DMR19: 2712001 | 43 | 1.433 | GNG7; DIRAS1; AC006538.2 | Signaling |
| DMR20: 29885001 | 5 | 0.5 | DUX4L37 | |
| DMRX: 119779001 | 21 | 2.1 | SNORA69; RPL39 | Translation |

In some embodiments, the methods further comprise determining whether a subject responds to a treatment. This may be used in a clinical test setting to quickly identify subjects that may respond to a certain treatment. In some embodiments, the treatment may be any hormone therapies that may increase sperm number and motility. The hormone therapy may be administering a therapeutically effective amount of follicle stimulating hormone (FSH), or an analog thereof to a subject. In some embodiments, the hormone therapy may be administering a therapeutically effective amount of human menopausal gonadotropin (hMG), or an analog thereof to the subject. In some embodiments, the treatment may comprise performing in vitro fertilization (IVF), intracytoplasmic sperm injection (ICSI), or any other suitable procedures that may result in successful pregnancy.

Methods of measuring methylation levels of DMRs in genomic DNA are well known to one skilled in the art. For example, microarray based methylome profiling and bioinformatics data analysis may be used to analyze DNA methylation profiles. In some embodiments, the microarray chip is a tiling array chip. In some embodiments, Methylated DNA immunoprecipitation (MeDIP) followed by next generation sequencing (NGS) is used. In some embodiments, MeDIP-Chip is used. Additional methods for detecting methylation levels can involve genomic sequencing before and after treatment of the DNA with bisulfite. When sodium bisulfite is contacted to DNA, unmethylated cytosine is converted to uracil, while methylated cytosine is not modified. Bisulfite methods may also be used in conjunction with pyrosequencing and PCR. Computer executable algorithms and software programs for implementing the same are also encompassed by the disclosure. Such software programs generally contain instructions for causing a computer to carry out the steps of the methods disclosed herein. The computer program will be embedded in a non-transient medium such as a hard drive, DVD, CD, thumb drive, etc.

Selection and identification of a subject for analysis may be predicated on and/or influenced by any number of factors. For example, the subject or subjects may be known or suspected to be afflicted with a disease or condition associated with infertility; or who have been or are suspected of having been exposed to an agent that causes, or is suspected of causing, infertility; or who have inexplicably inherited a disease or disease condition from a parent for which no DNA sequence mutation has been identified, etc. Subjects whose DNA is analyzed may be of any age, and in any stage of development, so long as cells containing a DNA sequence of interest can be obtained from the subject. For example, the subject may be an adult, an adolescent, a laboratory animal, etc. The cells from which the DNA is obtained may be any suitable cell, including but not limited to gametes, cells from swabs such as buccal swabs, cells sloughed into amniotic fluid, etc.

### Biomarkers

The different DMRs disclosed herein may be used as biomarkers for at least two related applications in fertility assessment. The panel of DMRs disclosed herein may serve as a sensitive and non-intrusive testing to diagnose whether a subject is infertile and screen for subjects that are responsive to any fertility or hormone treatment disclosed herein. In some embodiments, the panel of DMRs for indicating an infertility risk comprises at least 10, 20, 30, 40, 50, 60, 70, 80, 90, 100, or 200 DMRs listed in Table 2. The predictive value of a subject having infertility may increase as more DMRs listed in Table 2 are included in the panel. Diagnostic methods described herein for indicating a likelihood of infertility in a subject has a sensitivity that is greater than 75%, greater than 80%, greater than 85%, greater than 90%, greater than 95%, greater than 96%, greater than 97%, greater than 98%, greater than 99%, or about 100%. In some embodiments, the sensitivity is at least about 97%, 98%, 99%, or 99.5%.

In some embodiments, the panel of DMRs for indicating an infertility risk comprises at least 10, 20, 30, 40, or 50 DMRs listed in Table 3. The predictive value of a subject responding to a hormone treatment or fertility treatment may increase as more DMRs listed in Table 3 are included in the panel. Diagnostic methods described herein for determining responsiveness to a hormone treatment or fertility treatment in a subject has a sensitivity that is greater than 75%, greater than 80%, greater than 85%, greater than 90%, greater than 95%, greater than 96%, greater than 97%, greater than 98%, greater than 99%, or about 100%. In some embodiments, the sensitivity is at least about 97%, 98%, 99%, or 99.5%.

The genomic features described herein may be used in a variety of applications. For example, the DMRs of the disclosure can be indicative of having, the risk of having, or the risk of developing infertility or a condition that could lead to pregnancy complications and/or passage of heritable mutations to an infant. Thus the methods of the disclosure may be used, for example, in an in vitro fertilization clinic setting to test sperm for epimutations and for the potential to pass epigenetic information to offspring. The methods of the disclosure are also useful for screening potential sperm donors at a donation center. Further applications include screening applicants for health insurance coverage.

The detection of epigenetic modifications at the regions described herein (i.e. a positive diagnostic result) will suggest or confirm that the subject is infertile and treatments suitable for infertility can be instituted. For example, an appropriate infertility treatment, such as surgical extraction of sperm or FSH therapy, may be implemented. In other instances, a male subject may decide to utilize a sperm donor due to the subject's infertility or to prevent the possibility of pregnancy complications and/or the passage of heritable mutations to an infant attributable to the male subject.

Information concerning the type and extent of epigenetic modification in a subject may be used in a variety of decision making processes undertaken by a subject that is tested. For example, depending on the severity of the symptoms caused by an epigenetic modification that is identified, a subject may decide to forego having children or to terminate a pregnancy in order to prevent transmission of the modification to offspring. Diagnostic tests based on the present disclosure can be included in prenatal testing.

Thus, an aspect of the disclosure provides a method for treating a male subject who is infertile, comprising detecting the presence or absence of an epigenetic modification at one or more regions of at least one genomic DNA sequence or site obtained from a biological sample from said male subject, wherein said epigenetic modification comprises at least one differential DNA methylation region (DMR) listed in table 2; determining that said subject is infertile if said epigenetic modification is identified to be present in said at least one genomic DNA sequence or site; and administering an appropriate treatment protocol to said subject determined to be infertile.

In contrast, a negative result (no significant methylation level change at the site) suggests that the subject is not infertile and does not require an infertility treatment. Ongoing monitoring of the extent of epigenetic modification and methylation level of a site can provide valuable information regarding the outcome of the administration of agents (e.g. drugs or other therapies) which are intended to treat or prevent a condition caused by epimutation, i.e. the therapeutic responsiveness of a patient. Those of skill in the art will recognize that such analyses are generally carried out by comparing the results obtained using an unknown or experimental sample with results obtained a using suitable negative or positive controls, or both.

Subjects whose DNA is analyzed may be suffering from any of a variety of disorders (diseases, conditions, etc.) including but not limited to: various known late or adult onset conditions, such as low sperm production, infertility, abnormalities of sexual organs, kidney abnormalities, prostate disease, immune abnormalities, behavioral effects, etc. In other embodiments, no symptoms are present but screening using the diagnostics is employed to rule out the presence of “silent” epigenetic mutations which could cause disease symptoms in the future, or which could be inherited and cause deleterious effects in offspring.

The DMRs described herein may also be used to identify therapeutic modalities for the treatment of epigenetic mutations. Those of skill in the art will recognize that such methods of screening are typically carried out in vitro, e.g. using a DNA sequence that is immobilized in a vessel, or that is present in a cell. However, such tests may also be carried out in model laboratory animals. In one embodiment, candidate agents which reverse epigenetic modification are screened by analyzing the regions. In another embodiment, candidate agents which prevent epigenetic modifications are screened by analyzing the regions. In this way, the epigenetic biomarkers described herein can be used to facilitate, e.g. drug development and clinical trials patient stratification (i.e. pharmacoepigenomics).

Secondly, the DMRs described herein may also be used to identify responders and non-responders to FSH therapy. In men, FSH acts on the Sertoli cells of the testes to stimulate sperm production (spermatogenesis).

An embodiment of the disclosure provides a method of determining whether a male subject is a responder to FSH treatment comprising detecting the presence or absence of an epigenetic modification at one or more regions of at least one genomic DNA sequence obtained from a biological sample from said male subject, wherein said epigenetic modification comprises at least one differential DNA methylation region (DMR) listed in table 3; and determining that said subject is a responder to FSH treatment if said epigenetic modification is identified to be present in said at least one genomic DNA sequence or site; or determining that said subject is a non-responder to FSH treatment if said epigenetic modification is not identified to be present in said at least one genomic DNA sequence or site.

In some embodiments, FSH treatment is administered to the subject determined to be a responder to FSH treatment. In some embodiments, an infertility treatment other than FSH therapy is administered to the subject determined to be a non-responder, e.g. surgical extraction of sperm.

### Kits

In some embodiments, a kit is described. The kit comprises at least one polynucleotide that hybrid-izes to one of the DMR loci identified in table 2 or table 3 (or a nucleic acid sequence at least 90% identical to the DMR loci of table 2 or table 3), or that hybridizes to a region of DNA flanking one of the DMR loci identified in table 2 or table 3, and at least one reagent for detection of gene meth-ylation. Reagents for detection of methylation include, e.g., sodium bisulfite, polynucleotides de-signed to hybridize to sequence at or near the DMR loci of the disclosure if the sequence is not methylated, and/or a methylation-sensitive or methylation-dependent restriction enzyme. The kit may comprise bisulfite. The kits can provide solid supports in the form of an assay apparatus that is adapted to use in the assay. The kit may comprise a microarray chip or a DNA sequencing kit for sequencing any DMRs. The kit may further comprise detectable labels, optionally linked to a poly-nucleotide, e.g., a probe, in the kit. Other materials useful in the performance of the assays can also be included in the kit, including test tubes, transfer pipettes, and the like. The kit can also include written instructions for the use of one or more of these reagents in any of the assays described herein.

### Computer Systems

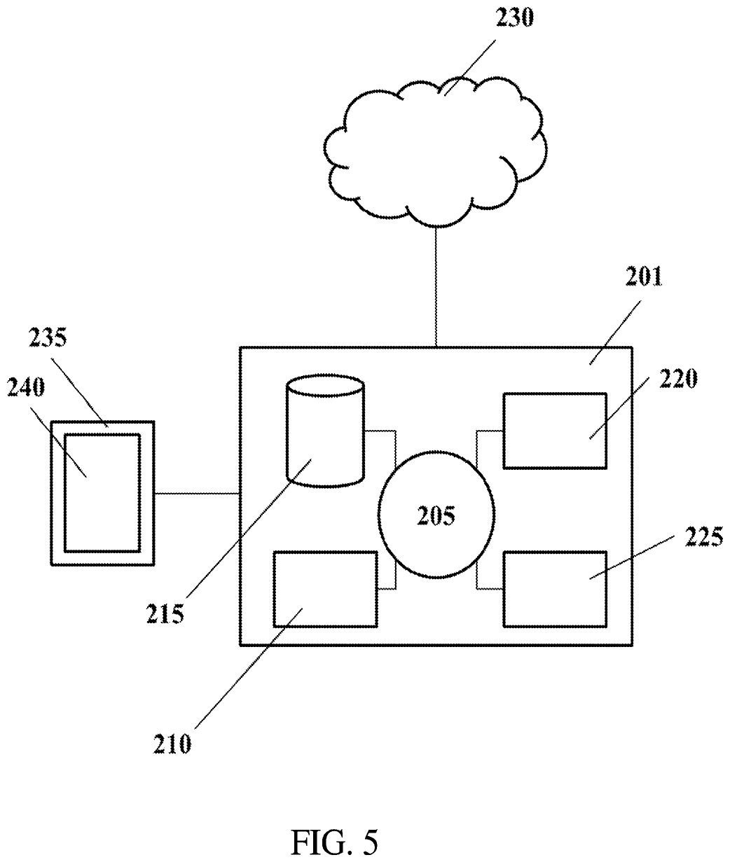

The present disclosure provides computer systems that are programmed to implement methods of the disclosure. FIG. 5 shows a computer system 201 that is programmed or otherwise configured to detect and measure methylation alteration and analyze epigenetic profile(s) in samples. The computer system 201 can regulate various aspects of the methods of the present disclosure, such as, for example, the extraction, detection, and/or sequencing of DNA in a sample. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, measurements of the reproductive hormone (e.g., DHEA, DHEA-S, estradiol, progesterone, testosterone, and/or AMH). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, determine the levels of DHEA, DHEA-S, estradiol, progesterone, testosterone, and/or AMH in a biological sample.

The computer processor may be further programmed to direct the assaying a nucleic acid sequence from at least a portion of a sperm sample from a subject. The computer processor may be further programmed to direct the detecting a methylation alteration of a portion of said nucleic acid sequence comprised in a differential DNA methylation region (DMR) listed in Table 2, thereby generating an epigenetic profile. The computer processor may be further programmed to direct the analyzing said epigenetic profile using a computer processor to compare said epigenetic profile with a reference epigenetic profile of a methylation level of corresponding portion(s) of said nucleic acid sequence comprised in said DMR listed in Table 2, wherein when said DMR is DMRMT:1, a second DMR is detected and analyzed.

The computer processor may be further programmed to transmit a result via a communication medium. In some embodiments, the result may comprise an epigenetic profile, a reference epigenetic profile, or both. In some embodiments, the result may comprise a likelihood whether the tested subject has a fertility issue, a likelihood whether the subject responds to a treatment disclosed herein, or both. In some embodiments, the result may comprise a recommendation for treating infertility.

Further, the computer processor may be further programmed to direct the assaying a nucleic acid sequence from at least a portion of a sperm sample from a subject. The computer processor may be further programmed to direct the detecting a methylation alteration of a portion of said nucleic acid sequence comprised in a differential DNA methylation region (DMR) listed in Table 3, thereby generating an epigenetic profile. The computer processor may be further programmed to direct the analyzing said epigenetic profile using a computer processor by comparing to a reference epigenetic profile of methylation status of corresponding portion(s) of said nucleic acid sequence comprised in said DMR listed in Table 3.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

### Example

The present example identifies a molecular biomarker or diagnostic for male infertility and provides the proof of concept that an epigenetic analysis is useful. Previously, an analysis for DNA methylation using a microarray of CpG islands and methylation sites constituting a couple percent of the human genome to identify altered methylation in sperm from infertility patients was performed. Observations are expanded in the current study with a genome-wide analysis that constitutes 95% of the human genome and advanced molecular analysis.

As disclosed herein, a genome-wide analysis of DNA methylation identifies a male infertility signature of DMRs that are present in male infertility patients. There was an efficient separation between the fertile versus infertile patient population with minimal overlap. A validation with a test set of infertile and fertile patients, not used in the initial establishment of the infertility DMRs, also efficiently separated the infertile versus the fertile patients. The infertility signature of DMRs was found in all the infertile patients' sperm samples showing the efficiency of the molecular biomarkers. The majority of the DNA methylation changes involved an increase in DNA methylation (i.e. hypermethylation), which suggests during early gametogenesis and/or spermatogenesis development of the sperm a hypermethylation may be an aspect of the male infertility molecular disease etiology. The development of a male infertility diagnostic is useful for the clinical management of the male infertility patient. Due to the increasing male infertility in the human population over the past fifty years, a greater demand for such an analysis in an assisted reproduction setting such as an IVF clinic is anticipated.

Observations also demonstrate that an epigenetic DNA methylation biomarker can identify pharmaceutical responders versus non-responders to FSH treatment among male infertility patients. The infertility responder versus non-responder DMR signature identified efficiently distinguished the two populations, and in contrast to the infertility diagnostic, the responder DMR signature involved an equal distribution of hypermethylation (increase) and hypomethylation (decrease) changes. No overlap was observed between the infertility DMRs and responder DMRs, suggesting a distinct set of epigenetic alterations. The current FSH therapeutic preparations in combination with this responder diagnostic allows for more effective patient management for infertility.

An initial sperm sample was collected upon enrollment, a second at the start of treatment, and a third after three months of treatment. Twenty-one patients were enrolled which included nine patients in the fertile control group and twelve in the infertility treatment group. The differences (mean±SD) between the seminal sample and hormonal parameters of both groups are shown in Table 1. Results from the baseline variables from the group of fertile subjects and those with infertility showed that there is a statistically significant difference in sperm number (i.e. concentration) between the fertile group and the infertile group, with the latter having the lowest values (95% CI-83, −2.87), p<0.001. Infertility patient samples also have a lower percentage of sperm motility, 95% CI [−2.62, 1.58], and p<0.001. The control group (fertile) showed lower FSHI levels than the infertility group, 95% CI [0.20, 0.95], p=0.005. Although not statistically significant, basal estradiol levels are higher in the group of subjects with infertility, 95% CI [−0.03, 0.89], p=0.06. Regarding the results in the infertility group after three months of FSH treatment (150 IU dose of FSH therapeutic three times per week), there was an increase in FSHI levels after treatment, although not statistically significant, p=0.063. However, the estimated confidence interval 95% difference should not be underestimated, 95% CI [−0.02, 0.73]. There was no statistically significant difference in regards to the group mean±SD in the other variables analyzed before and after treatment. In terms of pregnancy rate, there were three pregnancies ( 3/10, 30%). Two occurred after ICSI procedures, one was spontaneous, and seven non-pregnancies ( 7/10, 70%). There are two patients pending of ICSI procedure with frozen samples.

Table 1. Hormone, semen and sperm parameters. The mean±SD values for age (years), seminal volume (mL), sperm concentration (million/mL), motility (%), immotility (%), FSHI (IU/mL), LHI (IU/mL), estradiol (pg/mL), and testosterone (ng/mL).

TABLE 1
Mean hormone and semen parameters at baseline and after three months

| | Fertility | | Infertility | | Fertility | | Infertility | |
|---|---|---|---|---|---|---|---|---|
| | Control | | Treatment | | Control | | Treatment | |
| | baseline | | baseline | | 3 months | | 3 months | |
| | n = 9 | | n = 12 | | n = 9 | | n = 12 | |
| | Mean (±SD) | | Mean (±SD) | | Mean (±SD) | | Mean (±SD) | |
| | Median (1st, | | Median (1st, | | Median (1st, | | Median (1st, | |
| Variable | 3rd Q.) | | 3rd Q.) | | 3rd Q.) | | 3rd Q.) | |
| Age (years) | 39.11 | (3.02) | 35.83 | (4.15) | 39.11 | (3.02) | 35.83 | (4.24) |
| | 38 | (37, 40) | 35.5 | (33.75, 37.25) | 38 | (37, 40) | 35.5 | (33.75, 37.3) |
| Seminal vol (mL) | 3.12 | (1.59) | 2.73 | (1.39) | 2.82 | (1.71) | 2.93 | (1.22) |
| | 2.1 | (2, 4) | 3 | (1.8, 4) | 2 | (2, 3) | 3 | (2, 3) |
| Sperm concentration | 70 | (37.39) | 3.03 | (2.49) | 79.44 | (54.85) | 5.59 | (6.71) |
| (million/mL) | 50 | (43, 111.3) | 2 | (1, 4) | 55 | (40, 100) | 2.5 | (0.88, 10.25) |
| Motility (%) | 61.34 | (20.98) | 13.12 | (8.27) | 47.22 | (10.03) | 13.95 | (10.39) |
| | 55 | (45.8, 67) | 12.5 | (5, 20) | 45 | (40, 60) | 12.5 | (5.7, 20) |
| Immotility (%) | 38.66 | (20.98) | 86.88 | (8.27) | 52.78 | (10.03) | 86.05 | (10.39) |
| | 45 | (33, 54.2) | 87.5 | (80, 95) | 55 | (40, 60) | 87.5 | (80, 94.3) |
| FSH (IU/mL) | 3.01 | (0.7) | 5.79 | (2.64) | 3.33 | (1.16) | 7.97 | (3.18) |
| | 3.1 | (2.5, 3.4) | 5.5 | (3.6, 7.67) | 2.9 | (2.6, 4.1) | 7.75 | (5.67, 8.8) |
| LH (IU/mL) | 4.92 | (2.23) | 4.79 | (2.43) | 4.81 | (1.12) | 4.58 | (2.24) |
| | 4.6 | (4.3, 6.1) | 4.3 | (2.67, 6.85) | 4.9 | (4.1, 5.3) | 4.35 | (2.77, 5.35) |
| Estradiol (pg/mL) | 18.67 | (8.46) | 29.25 | (13.89) | 20.89 | (10.13) | 26.25 | (8.47) |
| | 16 | (12, 23) | 25 | (19, 37) | 21 | (16, 28) | 26.5 | (17.75, 32.3) |
| Total Testosterone | 4.99 | (1.4) | 4.9 | (1.45) | 4.99 | (1.61) | 5.01 | (1.49) |
| (ng/mL) | 4.75 | (4.2, 5.7) | 5.06 | (3.88, 5.76) | 4.7 | (3.84, 6.3) | 5.57 | (4.19, 5.84) |
| Bioavailable | 2.13 | (0.53) | 2.47 | (0.84) | 2.08 | (0.5) | 2.32 | (0.64) |
| testosterone | 2.06 | (1.9, 2.2) | 2.46 | (1.98, 2.77) | 1.9 | (1.75, 2.1) | 2.41 | (1.8, 2.71) |

(ng/mL)

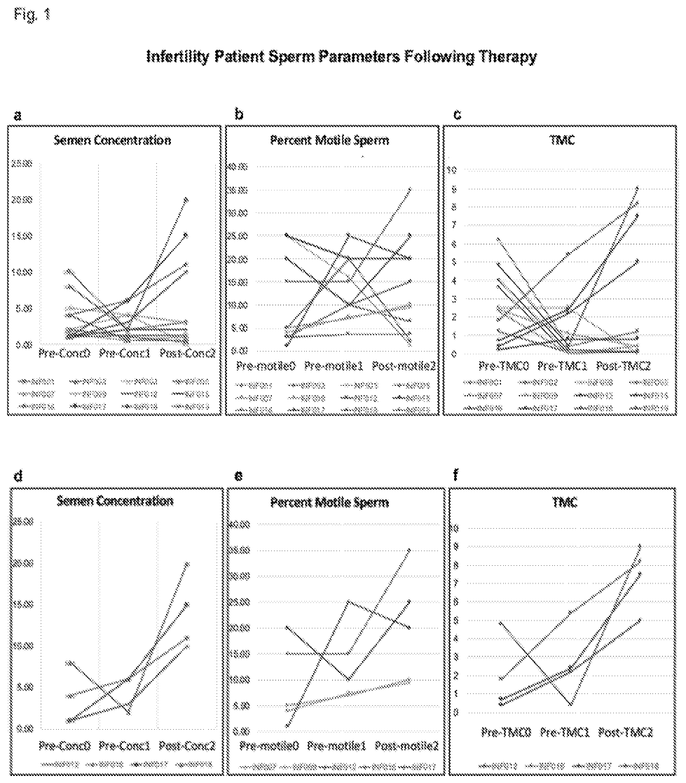

The fertile control and infertile treatment for baseline and after 3 months is presented with n-value indicated for each Individual patient information was used to identify the infertility patient responsiveness or non-responsiveness to FSH therapy. The infertility patients that show a 2-3 fold increase in sperm number (semen concentration) and/or motility following three month treatment are shown in FIGS. 1D, 1E, 1F, and were designated as responders. Although some variation occurred from the initial sperm sample collected at enrollment and second sample at the start of the FSH treatment, the final values following treatment were generally higher for all parameters in responder patients, and shown in FIGS. 1A-1F. The patients that responded to FSH therapy as shown in FIGS. 1C, 1D, and 1E were compared to the non-responsive patients as shown in FIGS. 1A, 1B, and 1C with the epigenetic analysis.

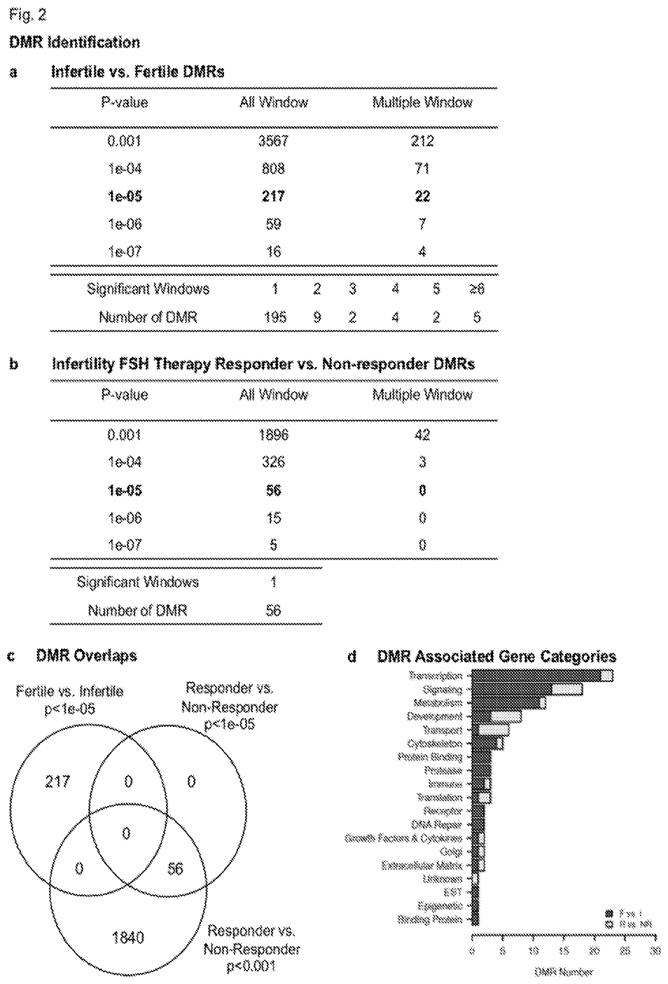

Individual patient samples from the initial sperm sample collected upon enrollment, the sample at the start of the FSH therapy treatment, and the sample after 3 months of treatment were prepared for epigenetic analysis. The DNA was extracted from the sperm then fragmented for a methylated DNA immunoprecipitation (MeDIP) analysis in order to identify differential DNA methylated regions (DMRs). The MeDIP is a genome-wide analysis examining 95% of the genome comprising low density CpG regions in comparison to the less than 5% of the genome of high density regions and CpG islands. The MeDIP DNA is then prepared for next generation DNA sequencing and bioinformatic analyses, as described in the Materials and Methods section. A comparison of the sequences derived from fertile versus infertile patient sperm identified DMRs for infertility assessment, as shown in FIG. 2A. At a p-value of p<1e-05 there were 217 DMRs identified, and the majority of these were within one 1000 bp windows with fewer having multiple 1000 bp windows involved. The DMRs at a number of different p-values are presented, but the p<1e⁻⁰⁵ was used for subsequent data analysis and a list of these DMRs are presented with various genomic features (Table 2). Therefore, a male infertility DMR signature was identified when comparing fertile versus infertile patients' sperm DNA.

All the infertility patients had a sperm collection prior to a three-month FSH therapeutic treatment period after which another sperm sample was collected for analysis. FIG. 2B showed comparison of sperm from the infertility patients who responded to FSH treatment versus those who did not respond identified DMRs associated with the responder patients. A variety of p-value DMR data is shown, and at p<1e⁻⁰⁵ there were 56 DMRs selected for subsequent data analysis. All the 56 DMRs had a single 1000 bp window that was statistically significant (p<1e⁻⁰⁵; FDR-adjusted p<0.1). A list of the responder DMRs and genomic features is presented in Table 3. An overlap analysis of the responder DMRs with the infertility DMRs demonstrated no overlap at p<1e⁻⁰⁵, as shown in FIG. 2C. The overlap analysis using a p<0.001 for the responder DMRs also shows no overlap with the infertility DMRs suggesting distinct epigenetic biomarkers. Approximately 50% of the DMRs have associated genes within 10 kb of a gene. The gene categories of these DMR associated genes are summarized in FIG. 2D. Surprisingly, the major categories of transcription, signaling, metabolism, transport and cytoskeleton are common between the infertility DMRs and responder DMRs. Therefore, an FSH therapeutic responder epigenetic biomarker (i.e. DMR signature) was identified when comparing infertility patient responder versus non-responder sperm.

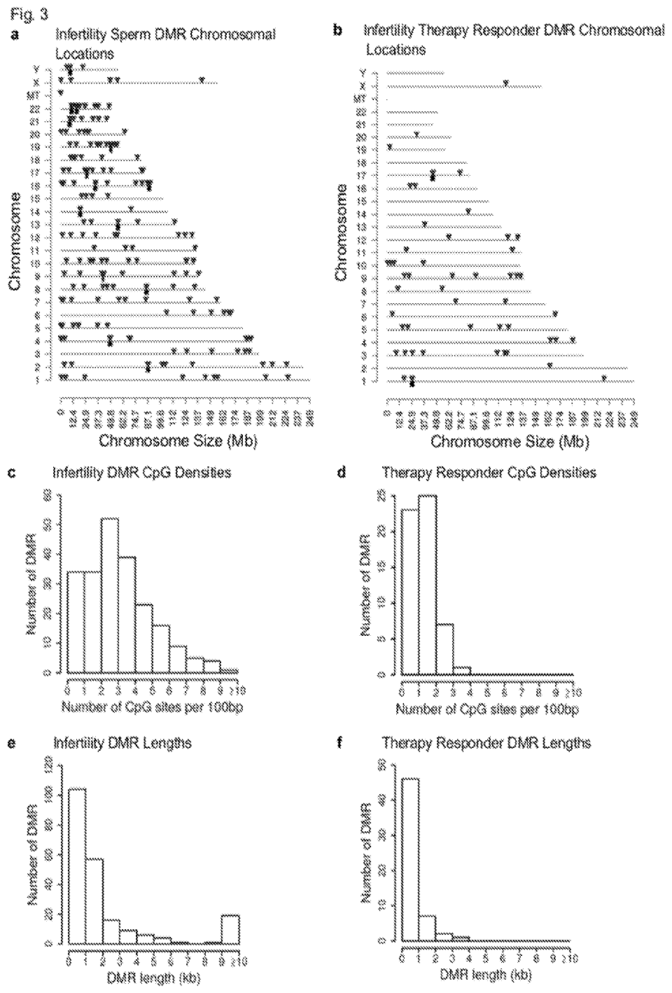

The genomic features of the infertility DMRs and FSH therapeutic responder DMRs were investigated. The chromosomal locations of the DMRs within the human genome are presented in FIGS. 3A and 3B. The arrowhead indicates an individual DMR and the box represents a cluster of DMRs. The infertility DMRs are present on all the chromosomes and mitochondrial DNA. The therapy responsiveness DMRs are also on most chromosomes. The CpG density where DNA methylation occurs is generally less than 10 CpG per 100 bp with 1-4 CpG predominant for the infertility and therapy response DMRs, as seen in FIGS. 3C and 3D. The size of the DMRs was predominantly 1-4 kb for the infertility DMRs and 1-2 kb for the therapy response DMRs, as shown in FIGS. 3E and 3F. Additional genomic features indicate approximately 90% of the infertility DMRs and 50% of the responder DMRs have an increase in DNA methylation and the rest a decrease in DNA methylation. Therefore, the majority of DMRs in infertility involve an increase in DNA methylation, while only half in the responder DMRs.

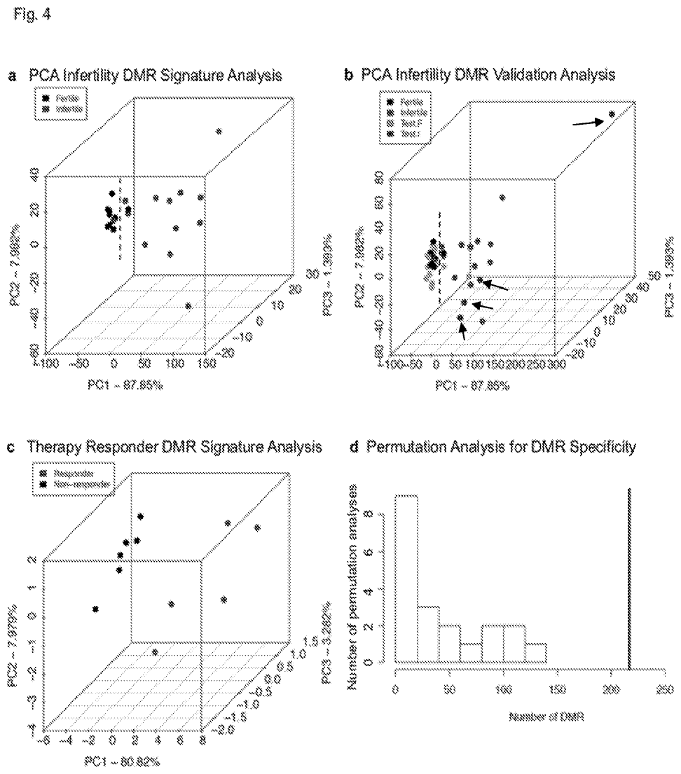

The statistical significance and associations of the DMRs for each comparison was investigated. A principal component analysis (PCA) of the infertility versus fertility DMR principal components is presented in FIG. 4A. There was a general clustering of the fertile DMRs and infertile DMRs from each other with only one DMR from each group outside the cluster. Therefore, good separation of the DMR in the PCA analysis was observed for the infertile versus fertile DMR groups. A validation set of samples collected that were selection failures due to a variety of reasons and not used in the infertility DMR analysis for DMR identification, as shown in FIG. 4A. However, the sperm samples collected were used to determine fertility and infertility parameters. These selection failure samples were used as a validation test set of samples and analyzed with the MeDIP-Seq procedure. These were included in a separate PCA analysis. The test infertility samples clustered with the infertility group, and majority of the test fertility samples clustered with the fertility group, as shown in FIG. 4B. Two of the test fertility samples clustered with the infertility group. A PCA analysis with this validation set demonstrates the green DMR fertile test set (the dots left of the dashed line) primarily associates with the fertility patients while all the blue DMR infertile test set samples (the dots right of the dashed line with arrows identified them) associate with the infertile group. This test set helps validate the infertility DMR signature identified in the current study. A similar PCA analysis of the FSH therapeutic responsiveness DMRs was performed. A clustering of the non-responsive DMRs was observed and all were distinct from the responsive cluster, as shown in FIG. 4C. No validation test set existed for the responsive DMR signature. A final permutation analysis was performed on the fertility versus infertility data to demonstrate the DMRs were not due to background variation and randomly generated. The permutation analysis demonstrates that the number of infertility DMRs generated from the comparison was significantly greater than the DMRs generated from random subsets within the analysis, as shown in FIG. 4D. The vertical line to the right indicates the comparison DMRs versus the low numbers from the random subset comparison.

### DISCUSSION

The current study was designed to identify a molecular biomarker or diagnostic for male infertility and provide that an epigenetic analysis will be useful. Previously, researchers utilized an analysis for DNA methylation using a microarray of CpG islands and methylation sites constituting a couple percent of the human genome to identify altered methylation in sperm from infertility patients. Observations are expanded in the current study with a genome wide analysis that constitutes 95% of the human genome and advanced molecular analysis.

Observations from the current study demonstrate a genome-wide analysis of DNA methylation identifies a male infertility signature of DMRs that are present in male infertility patients. There was an efficient separation between the fertile versus infertile patient population with minimal overlap. A validation with a test set of infertile and fertile patients, not used in the initial establishment of the infertility DMRs, also distinctively and efficiently separated the infertile versus the fertile patients. The infertility signature of DMRs was found in all the infertile patients' sperm samples showing the efficiency of the molecular biomarkers. The majority of the DNA methylation change involved an increase in DNA methylation (i.e. hypermethylation), which suggests during early gametogenesis and/or spermatogenesis development of the sperm a hypermethylation may be an aspect of the male infertility molecular disease etiology.

Observations also demonstrate that an epigenetic DNA methylation biomarker can be used to identify pharmaceutical responders versus non-responders to FSH treatment among male infertility patients. The infertility responder versus non-responder DMR signature identified efficiently distinguished the two populations, and in contrast to the infertility diagnostic, the responder DMR signature involved an equal distribution of hypermethylation (increase) and hypomethylation (decrease) changes. No overlap was observed between the infertility DMRs and responder DMRs, suggesting a distinct set of epigenetic alterations.

In conclusion, the current study identified a male infertility epigenetic DMR signature for use as a diagnostic, as well as an FSH therapy response diagnostic within this patient population. The advancement of such technology is anticipated to enhance the diagnosis and management of male infertility patients, as well as improve general therapeutic options and therapeutic development.

Materials and Methods

Clinical Sample Collection and Analysis

A single center (Urology Department at Hospital Universitari i Politècnic La Fe), prospective and open clinical study. The IRB approval code protocol 2015-002521-19. We included two groups (infertility vs fertility). The infertility men (inability of the couple to become pregnant after one year of sexual activity), included Caucasians between 25-45 years of age with a total sperm concentration (concentration in millions/mL×volume in mL) between 1-10 million (oligozoospermia) in at least 2 spermiograms obtained after a 2-4 day period of sexual abstinence and with a 7-day separation period between tests. The hormone profile used inclusion criteria of FSH 2-12 IU/mL, total testosterone>300 ng/mL and bioavailable testosterone (calculated with the Sexual Hormone Binding Globulin or SHBG albumin)>145 ng/dL. The fertile control group included Caucasians without vasectomy and had a child in the last five years with a sperm concentration and motility above the 50th percentile according to the parameters set forth in the 5th edition of the World Health Organization (WHO) guidelines in at least two spermiograms obtained after a 2-4 day period of sexual abstinence and with a 7-day period between tests. The hormones profiled used inclusion criteria of estradiol<50 pg/mL, FSH<4.5 IU/L, total testosterone>300 ng/dL and bioavailable testosterone>145 ng/dL.

Initial semen analysis and basal hormone determination to assess eligibility criteria were performed. Sperm samples were processed and stored for the subsequent epigenetic analysis. The infertility group received 150 IU of urinary or recombinant FSH three times per week for 12 weeks and the fertile control group did not received treatment. After three months of treatment, semen analysis and hormone profiles were retested in both groups. The sperm samples of three months with treatment for infertility and three months after for control group were processed and stored for the epigenetic test.

DNA Preparation

Frozen human sperm samples were stored at −20 C and thawed for analysis. Genomic DNA from sperm was prepared as follows: A minimum of a one hundred μl of sperm suspension was used then 820 μl DNA extraction buffer (50 mM Tris pH 8, 10 mM EDTA pH 8, 0.5% SDS) and 80 μl 0.1 M Dithiothreitol (DTT) was added and the sample incubated at 65 C for 15 minutes. 80 μl Proteinase K (20 mg/ml) was added and the sample incubated on a rotator at 55 C for at least 2 hours. After incubation, 300 μl of protein precipitation solution (Promega, A795A, Madison, Wis.) was added, the sample was mixed and incubated on ice for 15 minutes, then spun at 4 C at 13,000 rpm for 30 minutes. The supernatant was transferred to a fresh tube, then precipitated over night at −20 C with the same volume 100% isopropanol and 2 μl glycoblue. The sample was then centrifuged and the pellet was washed with 75% ethanol, then air-dried and resuspended in 100 μl H₂O. DNA concentration was measured using the Nanodrop (Thermo Fisher, Waltham, Mass.). The freeze-thaw will destroy any contaminating somatic cells within the sperm collection.

Methylated DNA Immunoprecipitation (MeDIP)

Methylated DNA Immunoprecipitation (MeDIP) with genomic DNA was performed as follows: individual sperm DNA samples were diluted to 130 μl with 1× Tris-EDTA (TE, 10 mM Tris, 1 mM EDTA) and sonicated with a COVARIS® M220 ultrasonicator using the 300 bp setting. Fragment size was verified on a 2% E-gel agarose gel. The sonicated DNA was transferred from the tube to a 1.7 ml microfuge tube and the volume was measured. The sonicated DNA was then diluted with TE buffer (10 mM Tris HCl, pH7.5; 1 mM EDTA) to 400 μl, heat-denatured for 10 min at 95 C, then immediately cooled on ice for 10 min. Then 100 μl of 5× IP buffer and 5 μg of antibody (monoclonal mouse anti 5-methyl cytidine; Diagenode #C15200006) were added to the denatured sonicated DNA. The DNA-antibody mixture was incubated overnight on a rotator at 4 C. The following day magnetic beads (DYNABEADS® M-280 Sheep anti-Mouse IgG; 11201D) were pre-washed as follows: The beads were resuspended in the vial, then the appropriate volume (50 μl per sample) was transferred to a microfuge tube. The same volume of Washing Buffer (at least 1 mL 1×PBS with 0.1% BSA and 2 mM EDTA) was added and the bead sample was resuspended. The tube was then placed into a magnetic rack for 1-2 minutes and the supernatant was discarded. The tube was removed from the magnetic rack and the beads were washed once. The washed beads were resuspended in the same volume of 1×IP buffer (50 mM sodium phosphate ph7.0, 700 mM NaCl, 0.25% TritonX-100) as the initial volume of beads. 50 μl of beads were added to the 500 μl of DNA-antibody mixture from the overnight incubation, then incubated for 2 h on a rotator at 4 C. After the incubation the bead-antibody-DNA complex was washed three times with 1×IP buffer as follows: The tube was placed into a magnetic rack for 1-2 minutes and the supernatant was discarded, then washed with 1×IP buffer 3 times. The washed bead-DNA solution was then resuspended in 250 μl digestion buffer with 3.5 μl Proteinase K (20 mg/ml). The sample was then incubated for 2-3 hours on a rotator at 55 C and then 250 gi of buffered Phenol-Chloroform-Isoamylalcohol solution was added to the sample and the tube was vortexed for 30 sec and then centrifuged at 14,000 rpm for 5 min at room temperature. The aqueous supernatant was carefully removed and transferred to a fresh microfuge tube. Then 250 μl chloroform were added to the supernatant from the previous step, vortexed for 30 sec and centrifuged at 14,000 rpm for 5 min at room temperature. The aqueous supernatant was removed and transferred to a fresh microfuge tube. To the supernatant 21 of glycoblue (20 mg/ml), 20 μl of 5M NaCl and 500 μl ethanol were added and mixed well, then precipitated in −20 C freezer for 1 hour to overnight. The precipitate was centrifuged at 14,000 rpm for 20 min at 4° C. and the supernatant was removed, while not disturbing the pellet. The pellet was washed with 500 μl cold 70% ethanol in −20 C freezer for 15 min. then centrifuged again at 14,000 rpm for 5 min at 4 C and the supernatant was discarded. The tube was spun again briefly to collect residual ethanol to the bottom of the tube and as much liquid as possible was removed with gel loading tip. The pellet was air-dried at RT until it looked dry (about 5 minutes) then resuspended in 20 μl H2O or TE. DNA concentration was measured in a QUBIT® fluorometer (Life Technologies) with ssDNA kit (Molecular Probes Q10212).

MeDIP-Seq Analysis

The MeDIP DNA samples were used to create libraries for next generation sequencing (NGS) using the NEBNEXT® ULTRATM RNA Library Prep Kit for ILLUMINA® (San Diego, Calif.) starting at step 1.4 of the manufacturer's protocol to generate double stranded DNA. After this step the manufacturer's protocol was followed. Each sample received a separate index primer. NGS was performed at WSU Spokane Genomics Core using the ILLUMINA HISEQ® 2500 high-throughput sequencing system with a PE50 application, with a read size of approximately 50 bp and approximately 20-25 million reads per sample and 9-10 sample libraries each were run in one lane.

Bioinformatics and Statistics

Basic read quality was verified using summaries produced by the FastQC program. Reads were filtered and trimmed to remove low quality base pairs using Trimmomatic. Samples with elevated read depths were randomly subsampled to obtain more consistent read depths across all samples. The reads for each sample were mapped to the GRCh38 human genome using Bowtie2 with default parameter options. The mapped read files were then converted to sorted BAM files using SAMtools. To identify DMR, the reference genome was broken into 1000 bp windows. The MEDIPS R package was used to calculate differential coverage between control and exposure sample groups. The edgeR p-value was used to determine the relative difference between the two groups for each genomic window. Windows with an edgeR p-value less than 10 were considered DMRs. The DMR edges were extended until no genomic window with an edgeR p-value less than 0.1 remained within 1000 bp of the DMR. CpG density and other information was then calculated for the DMR based on the reference genome. DMR were annotated using the biomaRt R package 31 to access the Ensembl database. The genes that overlapped with DMR were then input into the KEGG pathway search to identify associated pathways. The DMR associated genes were then sorted into functional groups using information provided by the DAVID and Panther databases incorporated into an internal curated database. All MeDIP-Seq genomic data obtained in the current study have been deposited in the NCBI public GEO database.

A permutation analysis to determine the significance of the number of DMR identified for each comparison was performed. For this analysis, samples from the two treatment groups were randomly assigned group membership. The number of samples in each treatment group was held constant. Twenty random permutations of each analysis were performed to obtain a null distribution for the expected number of DMR.

Statistical Analysis

In order to characterize clinical parameters of both groups (control and treatment group), a numerical descriptive analysis has been made using the mean with standard deviation (SD) and the median (1st and 3rd quartile). The baseline differences between the treatment group and the control group were then compared, as well as the effect of FSH between the before and after treatment in the treated group, in all variables collected. For this, we have used mixed linear regression models in case we had several measures per patient (semen volume and sperm concentration), and in the case of motility a beta logistic regression model was performed given its percentage character. The mixed models control the non-independence of data given that there are several measures per patient.

In the fertile group both baseline and 3-month measures were considered, because no difference was expected. On the other hand, in the infertile treatment group, two samples were extracted from these three variables (volume, concentration and motility). In this way, the power increases and there is a greater probability that we detect differences. In all other cases, associations between variables have been studied using linear regression models. The statistical analyses were performed with the statistical software R (version 3.4.1) and the packages nlme (version 3.1-131), lme4 (1.1-13), glmmADMB (0.8.3.3) and betareg (version 3.1-0). A p-value of less than 0.05 was considered statistically significant.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A method, comprising:
assaying a nucleic acid sequence from at least a portion of a sperm sample from a subject;
detecting a methylation alteration of at least a portion of said nucleic acid sequence comprised in a differential DNA methylation region (DMR) listed in Table 2, thereby generating an epigenetic profile; and
analyzing said epigenetic profile using a computer processor to compare said epigenetic profile with a reference epigenetic profile of a methylation level of at least a portion of a corresponding nucleic acid sequence comprised in said DMR listed in Table 2, wherein when said DMR is DMRMT:1, at least a portion of a nucleic acid sequence comprised in a second DMR, optionally listed in Table 2, is detected and analyzed.

2. The method of claim 1, further comprising determining a likelihood of fertility in said subject at least based in part on said analyzing.

3. The method of either claim 1 or claim 2, wherein said subject is infertile or has a reduced fertility relative to a normal subject.

4. The method of claim 3, further comprising administering a treatment to said subject.

5. The method of claim 4, wherein said treatment comprises performing in vitro fertilization (IVF).

6. The method of claim 4, wherein said treatment comprises performing intracytoplasmic sperm injection (ICSI).

7. The method of claim 4, wherein said treatment comprises administering a therapeutic effective amount of follicle stimulating hormone (FSH), or an analog thereof to said subject.

8. The method of claim 4, wherein said treatment comprises administering a therapeutic effective amount of human menopausal gonadotropin (hMG), or an analog thereof to said subject.

9. The method of any of claims 1-8, wherein said reference epigenetic profile comprises a methylation level of a nucleotide sequence of a fertile subject.

10. The method of any of claims 1-9, wherein said detecting comprises measuring an epigenetic alteration of: six or more, ten or more, fifteen or more, twenty or more, thirty or more, forty or more, fifty or more, sixty or more, seventy or more, eighty or more, ninety or more, or one hundred or more DMRs listed in Table 2.

11. The method of any of claims 1-9, wherein said detecting comprise measuring a methylation alteration of 1-217 DMRs listed in Table 2.

12. The method of any of claims 1-9, wherein said detecting comprise measuring a methylation alteration of 1-50 DMRs listed in Table 2.

13. The method of any of claims 1-9, wherein said detecting comprise measuring a methylation alteration of 100-217 DMRs listed in Table 2.

14. The method of any of claims 1-9, wherein said detecting comprise measuring a methylation alteration of 50-150 DMRs listed in Table 2.

15. A method, comprising:
assaying a nucleic acid sequence from at least a portion of a sperm sample from a subject;
detecting a methylation alteration of at least a portion of said nucleic acid sequence comprised in a differential DNA methylation region (DMR) listed in Table 3, thereby generating an epigenetic profile; and
analyzing said epigenetic profile using a computer processor by comparing to a reference epigenetic profile of methylation level of at least a portion of corresponding nucleic acid sequence comprised in said DMR listed in Table 3.

16. The method of claim 15, when administering a treatment, further comprising determining whether said subject responds to a treatment.

17. The method of claim 16, wherein said treatment comprises administering a therapeutic effective amount of follicle stimulating hormone (FSH), or an analog thereof to said subject.

18. The method of claim 16, wherein said treatment comprises administering a therapeutic effective amount of human menopausal gonadotropin (hMG), or an analog thereof to said subject.

19. The method of either of claim 17 or claim 18, wherein when said subject does not respond to said treatment, further comprising performing IVF.

20. The method of either of claim 17 or claim 18, wherein when said subject does not respond to said treatment, further comprising performing ICSI.

21. The method of claim 15, wherein said reference epigenetic profile comprises a methylation level of a nucleotide sequence of a subject that responds to said treatment.

22. The method of claim 21, wherein said subject has increased sperm number or sperm motility after receiving said treatment.

23. The method of any of claims 15-22, wherein said detecting comprises measuring an epigenetic alteration of: six or more, ten or more, fifteen or more, twenty or more, thirty or more, forty or more, fifty or more DMRs listed in Table 3.

24. The method of any of claims 15-22, wherein said detecting comprises measuring an epigenetic alteration of 1-56 DMRs listed in Table 3.

25. The method of any of claims 15-22, wherein said detecting comprises measuring an epigenetic alteration of 1-20 DMRs listed in Table 3.

26. The method of any of claims 15-22, wherein said detecting comprises measuring an epigenetic alteration of 30-56 DMRs listed in Table 3.

27. The method of any of claims 15-22, wherein said detecting comprises measuring an epigenetic alteration of 1-35 DMRs listed in Table 3.

28. The method of any of claims 1-27, wherein said assaying comprises performing a sequencing analysis, a pyrosequencing analysis, a microarray analysis, or any combination thereof.

29. The method of claim 28, wherein said sequencing analysis comprises a methylated DNA immunoprecipitation (MeDIP) sequencing.

30. The method of claim 29, wherein said MeDIP comprises using an antibody that binds to a methylated base (mB).

31. The method of claim 30, wherein said hmB is 5-methylated base (5-mB).

32. The method of claim 31, wherein said 5-hmB is a 5-methylated cytosine (5-mC).

33. The method of any of claims 1-32, wherein said epigenetic profile comprises an increased methylation level.

34. The method of any of claims 1-32, wherein said epigenetic profile comprises a decreased methylation level.

35. The method of any of claims 1-32, wherein said nucleotide sequence comprises a cytosine phosphate guanine (CpG) region.

36. The method of any of claims 1-32, wherein said DMRs listed either in Table 2 or Table 3 comprise a CpG density that is less than 10 CpG regions per 100 bp nucleotides.

37. The method of any of claims 1-32, wherein said DMRs listed either in Table 2 or Table 3 are produced from about 95% of a genome.

38. The method of any of claims 1-14, wherein said DMR listed in Table 2 has a range of about 1000 bp to about 50,000 bp nucleotide sequence.

39. The method of any of claims 1-14, wherein said DMR listed in Table 2 has a range of about 1000 bp to about 4000 bp nucleotide sequence

40. The method of any of claims 15-27, wherein said DMR listed in Table 3 has a range of about 1000 bp to about 5000 bp nucleotide sequence.

41. The method of any of claims 15-27, wherein said DMR listed in Table 3 has a range of about 1000 bp to about 2000 bp nucleotide sequence.

42. The method of any of claims 1-41, wherein Table 2 does not overlap with Table 3.

43. The method of any of claims 1-42, further comprising obtaining said sperm sample from said subject.

44. The method of any of claims 1-42, further comprising contacting said nucleic acid sequence with a 5-mC specific antibody.

45. The method of any of claims 1-42, further comprising contacting said nucleic acid sequence with a bisulfite.

46. The method of any of claims 1-45, wherein said subject is a human subject.

47. The method of any of claims 1-46, further comprising transmitting a result via a communication medium.

48. The method of claim 47, wherein said result comprises an epigenetic profile, a reference epigenetic profile, or both.

49. A kit, comprising:
bisulfite;
a plurality of primers configured to detect a differential DNA methylation region (DMR) listed in Table 2 or Table 3; and
a microarray chip or a DNA sequencing kit.

50. A computer-readable medium comprising machine-executable code that, upon execution by a computer processor, implements a method for determining a likelihood of fertility in a subject, comprising:
assaying a nucleic acid sequence from at least a portion of a sperm sample from a subject;
detecting a methylation alteration of at least a portion of said nucleic acid sequence comprised in a differential DNA methylation region (DMR) listed in Table 2, thereby generating an epigenetic profile; and
analyzing said epigenetic profile using a computer processor to compare said epigenetic profile with a reference epigenetic profile of a methylation level of at least a portion of corresponding nucleic acid sequence comprised in said DMR listed in Table 2, wherein when said DMR is DMRMT:1, at least a portion of a nucleic acid sequence comprised in a second DMR, optionally listed in Table 2, is optionally detected, and is analyzed.

51. A computer-readable medium comprising machine-executable code that, upon execution by a computer processor, implements a method for determining whether a subject responds to a treatment, comprising:
assaying a nucleic acid sequence from at least a portion of a sperm sample from a subject;
detecting a methylation alteration of at least a portion of said nucleic acid sequence comprised in a differential DNA methylation region (DMR) listed in Table 3, thereby generating an epigenetic profile; and
analyzing said epigenetic profile using a computer processor by comparing to a reference epigenetic profile of methylation status of at least a portion of corresponding nucleic acid sequence comprised in said DMR listed in Table 3.
